SEQUENCE LISTING (45 sequences)

SEQ ID NO: 1 - protein, 390 aa, Homo sapiens
MRWRTILLQYCFLLITCLLTALEAVPIDIDKTKVQNIHPVESAKIEPPDTGLYYDEYLKQVIDVLETDKHFREKLQKADIEEIKSGRLSKELDLVSHHVRTKLDELKRQEVGRLRMLIKAKLDSLQDIGMDHQALLKQFDHLNHLNPDKFESTDLDMLIKAATSDLEHYDKTRHEEFKKYEMMKEHERREYLKTLNEEKRKEEESKFEEMKKKHENHPKVNHPGSKDQLKEVWEETDGLDPNDFDPKTFFKLHDVNSDGFLDEQELEALFTKELEKVYDPKNEEDDMVEMEEERLRMREHVMNETLDQQQFFTEEELKEYENIIALQENELKKKADELQKQKEELQRQHDQLEAQKLEYHQVIQQMEQKKLQQGIPPSGPAGELKFEPHI

SEQ ID NO: 2 - protein, 87 aa, Homo sapiens
MSQAEFEKAAEEVRHLKTKPSDEEMLFIYGHYKQATVGDINTERPGMLDFTGKAKWDAWNELKGTSKEDAMKAYINKVEELKKKYGI

SEQ ID NO: 3 - protein, 98 aa, Homo sapiens
MMCGAPSATQPATAETQHIADQVRSQLEEKENKKFPVFKAVSFKSQVVAGTNYFIKVHVGDEDFVHLRVFQSLPHENKPLTLSNYQTNKAKHDELTYF

SEQ ID NO: 4 - protein, 375 aa, Homo sapiens
MERASCLLLLLLPLVHVSATTPEPCELDDEDFRCVCNFSEPQPDWSEAFQCVSAVEVEIHAGGLNLEPFLKRVDADADPRQYADTVKALRVRRLTVGAAQVPAQLLVGALRVLAYSRLKELTLEDLKITGTMPPLPLEATGLALSSLRLRNVSWATGRSWLAELQQWLKPGLKVLSIAQAHSPAFSCEQVRAFPALTSLDLSDNPGLGERGLMAALCPHKFPAIQNLALRNTGMETPTGVCAALAAAGVQPHSLDLSHNSLRATVNPSAPRCMWSSALNSLNLSFAGLEQVPKGLPAKLRVLDLSCNRLNRAPQPDELPEVDNLTLDGNPFLVPGTALPHEGSMNSGVVPACARSTLSVGVSGTLVLLQGARGFA

SEQ ID NO: 5 - protein, 617 aa, Homo sapiens
MAEAKTHWLGAALSLIPLIFLISGAEAASFQRNQLLQKEPDLRLENVQKFPSPEMIRALEYIENLRQQAHKEESSPDYNPYQGVSVPLQQKENGDESHLPERDSLSEEDWMRIILEALRQAENEPQSAPKENKPYALNSEKNFPMDMSDDYETQQWPERKLKHMQFPPMYEENSRDNPFKRTNEIVEEQYTPQSLATLESVFQELGKLTGPNNQKRERMDEEQKLYTDDEDDIYKANNIAYEDVVGGEDWNPVEEKIESQTQEEVRDSKENIEKNEQINDEMKRSGQLGIQEEDLRKESKDQLSDDVSKVIAYLKRLVNAAGSGRLQNGQNGERATRLFEKPLDSQSIYQLIEISRNLQIPPEDLIEMLKTGEKPNGSVEPERELDLPVDLDDISEADLDHPDLFQNRMLSKSGYPKTPGRAGTEALPDGLSVEDILNLLGMESAANQKTSYFPNPYNQEKVLPRLPYGAGRSRSNQLPKAAWIPHVENRQMAYENLNDKDQELGEYLARMLVKYPEIINSNQVKRVPGQGSSEDDLQEEEQIEQAIKEHLNQGSSQETDKLAPVSKRFPVGPPKNDDTPNRQYWDEDLLMKVLEYLNQEKAEKGREHIAKRAMENM

SEQ ID NO: 6 - protein, 172 aa, Homo sapiens
MATWALLLLAAMLLGNPGLEVSVSPKGKNTSGRESGFGWAIWMEGLVFSRLSPEYYDLARAHLRDEEKSCPCLAQEGPQGDLLTKTQELGRDYRTCLTIVQKLKKMVDKPTQRSVSNAATRVCRTGRSRWRDVCRNFMRRYQSRVTQGLVAGETAQQICEDLRLCIPSTGPL

SEQ ID NO: 7 - protein, 478 aa, Homo sapiens
MIRKKRKRSAPPGPCRSHGPRPATAPAPPPSPEPTRPAWTGMGLRAAPSSAAAAAAEVEQRRSPGLCPPPLELLLLLLFSLGLLHAGDCQQPAQCRIQKCTTDFVSLTSHLNSAVDGFDSEFCKALRAYAGCTQRTSKACRGNLVYHSAVLGISDLMSQRNCSKDGPTSSTNPEVTHDPCNYHSHAGAREHRRGDQNPPSYLFCGLFGDPHLRTFKDNFQTCKVEGAWPLIDNNYLSVQVTNVPVVPGSSATATNKITIIFKAHHECTDQKVYQAVTDDLPAAFVDGTTSGGDSDAKSLRIVERESGHYVEMHARYIGTTVFVRQVGRYLTLAIRMPEDLAMSYEESQDLQLCVNGCPLSERIDDGQGQVSAILGHSLPRTSLVQAWPGYTLETANTQCHEKMPVKDIYFQSCVFDLLTTGDANFTAAAHSALEDVEALHPRKERWHIFPSSGNGTPRGGSDLSVSLGLTCLILIVFL

SEQ ID NO: 8 - protein, 98 aa, Homo sapiens
MILLPQPPKKLGLQENGELCMNSAQCKSNCCQHSSALGLARCTSMASENSECSVKTLYGIYYKCPCERGLTCEGDKTIVGSITNTNFGICHDAGRSKQ

SEQ ID NO: 9 - protein, 98 aa, Homo sapiens
MNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 10 - protein, 850 aa, Homo sapiens
MTSHYVIAIFALMSSCLATAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTVMGIAFAAFVIGALLTGALWYIYSHTGETAGRQQVPTSPPASENSSAAHSIGSTQSTPCSSSSTA

SEQ ID NO: 11 - protein, 291 aa, Homo sapiens
MQRARPTLWAAALTLLVLLRGPPVARAGASSAGLGPVVRCEPCDARALAQCAPPPAVCAELVREPGCGCCLTCALSEGQPCGIYTERCGSGLRCQPSPDEARPLQALLDGRGLCVNASAVSRLRAYLLPAPPAPGNASESEEDRSAGSVESPSVSSTHRVSDPKFHPLHSKIIIIKKGHAKDSQRYKVDYESQSTDTQNFSSESKRETEYGPCRREMEDTLNHLKFLNVLSPRGVHIPNCDKKGFYKKKQCRPSKGRKRGFCWCVDKYGQPLPGYTTKGKEDVHCYSMQSK

SEQ ID NO: 12 - protein, 484 aa, Homo sapiens [sequence omitted]

SEQ ID NO: 13 - protein, 445 aa, Homo sapiens
MLPWTALGLALSLRLALARSGAERGPPASAPRGDLMFLLDSSASVSHYEFSRVREFVGQLVAPLPLGTGALRASLVHVGSRPYTEFPFGQHSSGEAAQDAVRASAQRMGDTHTGLALVYAKEQLFAEASGARPGVPKVLVWVTDGGSSDPVGPPMQELKDLGVTVFIVSTGRGNFLELSAAASAPAEKHLHFVDVDDLHIIVQELRGSILDAMRPQQLHATEITSSGFRLAWPPLLTADSGYYVLELVPSAQPGAARRQQLPGNATDWIWAGLDPDTDYDVALVPESNVRLLRPQILRVRTRPGEAGPGASGPESGAGPAPTQLAALPAPEEAGPERIVISHARPRSLRVSWAPALGSAAALGYHVQFGPLRGGEAQRVEVPAGRNCTTLQGLAPGTAYLVTVTAAFRSGRESALSAKACTPDGPRPRPRPVPRAPTPGTASREP

SEQ ID NO: 14 - protein, 402 aa, Homo sapiens [sequence omitted]

SEQ ID NO: 15 - protein, 728 aa, Homo sapiens
MWVTKLLPALLLQHVLLHLLLLPIAIPYAEGQRKRRNTIHEFKKSAKTTLIKIDPALKIKTKKVNTADQCANRCTRNKGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRNPDDDAHGPWCYTGNPLIPWDYCPISRCEGDTTPTIVNLDHPVISCAKTKQLRVVNGIPTRTNIGWMVSLRYRNKHICGGSLIKESWVLTARQCFPSRDLKDYEAWLGIHDVHGRGDEKCKQVLNVSQLVYGPEGSDLVLMKLARPAVLDDFVSTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKIILTYKVPQS

SEQ ID NO: 16 - protein, 194 aa, Homo sapiens
MTMRHNWTPDLSPLWVLLLCAHVVTLLVRATPVSQTTTAATASVRSTKDPCPSQPPVFPAAKQCPALEVTWPEVEVPLNGTLSLSCVACSRFPNFSILYWLGNGSFIEHLPGRLWEGSTSRERGSTGTQLCKALVLEQLTPALHSTNFSCVLVDPEQVVQRHVVLAQLWAGLRATLPPTQEALPSSHSSPQQQG

SEQ ID NO: 17 - protein, 358 aa, Homo sapiens
MEQWDHFHNQQEDTDSCSESVKFDARSMTALLPPNPKNSPSLQEKLKSFKAALIALYLLVFAVLIPLIGIVAAQLLKWETKNCSVSSTNANDITQSLTGKGNDSEEEMRFQEVFMEHMSNMEKRIQHILDMEANLMDTEHFQNFSMTTDQRFNDILLQLSTLFSSVQGHGNAIDEISKSLISLNTTLLDLQLNIENLNGKIQENTFKQQEEISKLEERVYNVSAEIMAMKEEQVHLEQEIKGEVKVLNNITNDLRLKDWEHSQTLRNITLIQGPPGPPGEKGDRGPTGESGPRGFPGPIGPPGLKGDRGAIGFPGSRGLPGYAGRPGNSGPKGQKGEKGSGNTLRPVQLTDHIRAGPS

SEQ ID NO: 18 - protein, 219 aa, Homo sapiens
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRAERHVKEDDGRKSPGEVPPGTSPACILATWPPGLLVLLWQETTLPEHCFSWTLEAGTG

SEQ ID NO: 19 - protein, 461 aa, Homo sapiens
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 20 - protein, 281 aa, Homo sapiens
MGHPPLLPLLLLLHTCVPASWGLRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEGRPKDDRHLRGCGYLPGCPGSNGFHNNDTFHFLKCCNTTKCNEGPILELENLPQNGRQCYSCKGNSTHGCSSEETFLIDCRGPMNQCLVATGTHERSLWGSWLPCKSTTALRPPCCEEAQATHV

SEQ ID NO: 21 - protein, 282 aa, Homo sapiens
MALVLILQLLTLWPLCHTDITPSVPPASYHPKPWLGAQPATVVTPGVNVTLRCRAPQPAWRFGLFKPGEIAPLLFRDVSSELAEFFLEEVTPAQGGSYRCCYRRPDWGPGVWSQPSDVLELLVTEELPRPSLVALPGPVVGPGANVSLRCAGRLRNMSFVLYREGVAAPLQYRHSAQPWADFTLLGARAPGTYSCYYHTPSAPYVLSQRSEVLVISWEGEGPEARPASSAPGMQAPGPPPSDPGAQAPSLSSFRPRGLVLQPLLPQTQDSWDPAPPPSDPGV

SEQ ID NO: 22 - protein, 303 aa, Homo sapiens
MGRPLLLPLLPLLLPPAFLQPSGSTGSGPSYLYGVTQPKHLSASMGGSVEIPFSFYYPWELATAPDVRISWRRGHFHRQSFYSTRPPSIHKDYVNRLFLNWTEGQKSGFLRISNLQKQDQSVYFCRVELDTRSSGRQQWQSIEGTKLSITQAVTTTTQRPSSMTTTWRLSSTTTTTGLRVTQGKRRSDSWHISLETAVGVAVAVTVLGIMILGLICLLRWRRRKGQQRTKATTPAREPFQNTEEPYENIRNEGQNTDPKLNPKDDGIVYASLALSSSTSPRAPPSHRPLKSPQNETLYSVLKA

SEQ ID NO: 23 - protein, 263 aa, Homo sapiens
MAPRARRRRPLFALLLLCALLARLQVALQIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSSSLPARKPPNEPHVYLPGLIILLLFASVALVAAIIFGVCYRKKGKALTANLWHWINEACGRLSGDKEM

SEQ ID NO: 24 - protein, 368 aa, Homo sapiens
MSGAPTAGAALMLCAATAVLLSAQGGPVQSKSPRFASWDEMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDHKHLDHEVAKPARRKRLPEMAQPVDPAHNVSRLHHGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 25 - protein, 465 aa, Homo sapiens
MTSMASLFSFTSPAVKRLLGWKQGDEEEKWAEKAVDALVKKLKKKKGAMEELEKALSSPGQPSKCVTIPRSLDGRLQVSHRKGLPHVIYCRVWRWPDLQSHHELKPLDICEFPFGSKQKEVCINPYHYKRVESPVLPPVLVPRHNEFNPQHSLLVQFRNLSHNEPHMPQNATFPDSFHQPNNTPFPLSPNSPYPPSPASSTYPNSPASSGPGSPFQLPADTPPPAYMPPDDQMGQDNSQPMDTSNNMIPQIMPSISSRDVQPVAYEEPKHWCSIVYYELNNRVGEAFHASSTSVLVDGFTDPSNNKSRFCLGLLSNVNRNSTIENTRRHIGKGVHLYYVGGEVYAECLSDSSIFVQSRNCNFHHGFHPTTVCKIPSSCSLKIFNNQEFAQLLAQSVNHGFEAVYELTKMCTIRMSFVKGWGAEYHRQDVTSTPCWIEIHLHGPLQWLDKVLTQMGSPLNPISSVS

SEQ ID NO: 26 - protein, 129 aa, Homo sapiens
MARGSLRRLLRLLVLGLWLALLRSVAGEQAPGTAPCSRGSSWSADLDKCMDCASCRARPHSDFCLGCAAAPPAPFRLLWPILGGALSLTFVLGLLSGFLVWRRCRRREKFTTPIEETGGEGCPAVALIQ

SEQ ID NO: 27 - protein, 446 aa, Homo sapiens
MLLPQLCWLPLLAGLLPPVPAQKFSALTFLRVDQDKDKDCSLDCAGSPQKPLCASDGRTFLSRCEFQRAKCKDPQLEIAYRGNCKDVSRCVAERKYTQEQARKEFQQVFIPECNDDGTYSQVQCHSYTGYCWCVTPNGRPISGTAVAHKTPRCPGSVNEKLPQREGTGKTDDAAAPALETQPQGDEEDIASRYPTLWTEQVKSRQNKTNKNSVSSCDQEHQSALEEAKQPKNDNVVIPECAHGGLYKPVQCHPSTGYCWCVLVDTGRPIPGTSTRYEQPKCDNTARAHPAKARDLYKGRQLQGCPGAKKHEFLTSVLDALSTDMVHAASDPSSSSGRLSEPDPSHTLEERVVHWYFKLLDKNSSGDIGKKEIKPFKRFLRKKSKPKKCVKKFVEYCDVNNDKSISVQELMGCLGVAKEDGKADTKKRHTPRGHAESTSNRQPRKQG

SEQ ID NO: 28 - protein, 455 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 29 - protein, 406 aa, Homo sapiens
MIHTNLKKKFSCCVLVFLLFAVICVWKEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 30 - protein, 269 aa, Homo sapiens
MSPHPTALLGLVLCLAQTIHTQEDLPRPSISAEPGTVIPLGSHVTFVCRGPVGVQTFRLERDSRSTYNDTEDVSQASPSESEARFRIDSVREGNAGLYRCIYYKPPKWSEQSDYLELLVKGPTQRPSDNSHNEHAPASQGLKAEHLYILIGVSVVFLFCLLLLVLFCLHRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 31 - protein, 160 aa, Homo sapiens
MHRRRSRSCREDQKPVMDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQSHWNWRTRLLGWV

SEQ ID NO: 32 - protein, 492 aa, Homo sapiens [sequence omitted]

SEQ ID NO: 33 - protein, 776 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSATKQVQRRPPPAGPRSERGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 34 - protein, 344 aa, Homo sapiens
MAISKVFELGLVAGLGHQEFARPSRGYLAFRDDSIWPQEEPAIRPRSSQRVPPMGIQHSKELNRTCCLNGGTCMLGSFCACPPSFYGRNCEHDVRKENCGSVPHDTWLPKKCSLCKCWHGQLRCFPQAFLPGCDGLVMDEHLVASRTPELPPSARTTTFMLVGICLSIQSYYMAISKVFELGLVAGLGHQEFARPSRGYLAFRDDSIWPQEEPAIRPRSSQRVPPMGIQHSKELNRTCCLNGGTCMLGSFCACPPSFYGRNCEHDVRKENCGSVPHDTWLPKKCSLCKCWHGQLRCFPQAFLPGCDGLVMDEHLVASRTPELPPSARTTTFMLVGICLSIQSYY

SEQ ID NO: 35 - protein, 178 aa, Homo sapiens
MPPLHKTVVYTGEPQRSRLGADVCAVLRLSGPLKEQYAQEHGLNFQRLLDTSTYKEAFRKDMIRWGEEKRQADPGFFCRKIVEGISQPIWLVSDTRRVSDIQWFREAYGAVTQTVRVVALEQSRQQRGWVFTPGVDDAESECGLDNFGDFDWVIENHGVEQRLEEQLENLIEFIRSRL

SEQ ID NO: 36 - protein, 1040 aa, Homo sapiens
MGTATRRKPHLLLVAAVALVSSSAWSSALGSQTTFGPVFEDQPLSVLFPEESTEEQVLLACRARASPPATYRWKMNGTEMKLEPGSRHQLVGGNLVIMNPTKAQDAGVYQCLASNPVGTVVSREAILRFGFLQEFSKEERDPVKAHEGWGVMLPCNPPAHYPGLSYRWLLNEFPNFIPTDGRHFVSQTTGNLYIARTNASDLGNYSCLATSHMDFSTKSVFSKFAQLNLAAEDTRLFAPSIKARFPAETYALVGQQVTLECFAFGNPVPRIKWRKVDGSLSPQWTTAEPTLQIPSVSFEDEGTYECEAENSKGRDTVQGRIIVQAQPEWLKVISDTEADIGSNLRWGCAAAGKPRPTVRWLRNGEPLASQNRVEVLAGDLRFSKLSLEDSGMYQCVAENKHGTIYASAELAVQALAPDFRLNPVRRLIPAARGGEILIPCQPRAAPKAVVLWSKGTEILVNSSRVTVTPDGTLIIRNISRSDEGKYTCFAENFMGKANSTGILSVRDATKITLAPSSADINLGDNLTLQCHASHDPTMDLTFTWTLDDFPIDFDKPGGHYRRTNVKETIGDLTILNAQLRHGGKYTCMAQTVVDSASKEATVLVRGPPGPPGGVVVRDIGDTTIQLSWSRGFDNHSPIAKYTLQARTPPAGKWKQVRTNPANIEGNAETAQVLGLTPWMDYEFRVIASNILGTGEPSGPSSKIRTREAAPSVAPSGLSGGGGAPGELIVNWTPMSREYQNGDGFGYLLSFRRQGSTHWQTARVPGADAQYFVYSNESVRPYTPFEVKIRSYNRRGDGPESLTALVYSAEEEPRVAPTKVWAKGVSSSEMNVTWEPVQQDMNGILLGYEIRYWKAGDKEAAADRVRTAGLDTSARVSGLHPNTKYHVTVRAYNRAGTGPASPSANATTMKPPPRRPPGNISWTFSSSSLSIKWDPVVPFRNESAVTGYKMLYQNDLHLTPTLHLTGKNWIEIPVPEDIGHALVQIRTTGPGGDGIPAEVHIVRNGGTSMMVENMAVRPAPHPGTVISHSVAMLILIGSLEL

SEQ ID NO: 37 - protein, 453 aa, Homo sapiens
MGVLGRVLLWLQLCALTQAVSKLWVPNTDFDVAANWSQNRTPCAGGAVEFPADKMVSVLVQEGHAVSDMLLPLDGELVLASGAGFGVSDVGSHLDCGAGEPAVFRDSDRFSWHDPHLWRSGDEAPGLFFVDAERVPCRHDDVFFPPSASFRVGLGPGASPVRVRSISALGRTFTRDEDLAVFLASRAGRLRFHGPGALSVGPEDCADPSGCVCGNAEAQPWICAALLQPLGGRCPQAACHSALRPQGQCCDLCGAVVLLTHGPAFDLERYRARILDTFLGLPQYHGLQVAVSKVPRSSRLREADTEIQVVLVENGPETGGAGRLARALLADVAENGEALGVLEATMRESGAHVWGSSAAGLAGGVAAAVLLALLVLLVAPPLLRRAGRLRWRRHEAAAPAGAPLGFRNPVFDVTASEELPLPRRLSLVPKAAADSTSHSYFVNPLFAGAEAEA

SEQ ID NO: 38 - protein, 630 aa, Homo sapiens
METTPLNSQKQLSACEDGEDCQENGVLQKVVPTPGDKVESGQISNGYSAVPSPGAGDDTRHSIPATTTTLVAELHQGERETWGKKVDFLLSVIGYAVDLGNVWRFPYICYQNGGGAFLLPYTIMAIFGGIPLFYMELALGQYHRNGCISIWRKICPIFKGIGYAICIIAFYIASYYNTIMAWALYYLISSFTDQLPWTSCKNSWNTGNCTNYFSEDNITWTLHSTSPAEEFYTRHVLQIHRSKGLQDLGGISWQLALCIMLIFTVIYFSIWKGVKTSGKVVWVTATFPYIILSVLLVRGATLPGAWRGVLFYLKPNWQKLLETGVWIDAAAQIFFSLGPGFGVLLAFASYNKFNNNCYQDALVTSVVNCMTSFVSGFVIFTVLGYMAEMRNEDVSEVAKDAGPSLLFITYAEAIANMPASTFFAIIFFLMLITLGLDSTFAGLEGVITAVLDEFPHVWAKRRERFVLAVVITCFFGSLVTLTFGGAYVVKLLEEYATGPAVLTVALIEAVAVSWFYGITQFCRDVKEMLGFSPGWFWRICWVAISPLFLLFIICSFLMSPPQLRLFQYNYPYWSIILGYCIGTSSFICIPTYIAYRLIITPGTFKERIIKSITPETPTEIPCGDIRLNAV

SEQ ID NO: 39 - protein, 1117 aa, Homo sapiens
METFFRRHFRGKVPGPGEGQQRPSSVGLPTGKARRRSPAGQASSSLAQRRRSSAQLQGCLLSCGVRAQGSSRRRSSTVPPSCNPRFIVDKVLTPQPTTVGAQLLGAPLLLTGLVGMNEEEGVQEDVVAEASSAIQPGTKTPGPPPPRGAQPLLPLPRYLRRASSHLLPADAVYDHALWGLHGYYRRLSQRRPSGQHPGPGGRRASGTTAGTMLPTRVRPLSRRRQVALRRKAAGPQAWSALLAKAITKSGLQHLAPPPPTPGAPCSESERQIRSTVDWSESATYGEHIWFETNVSGDFCYVGEQYCVARMLKSVSRRKCAACKIVVHTPCIEQLEKINFRCKPSFRESGSRNVREPTFVRHHWVHRRRQDGKCRHCGKGFQQKFTFHSKEIVAISCSWCKQAYHSKVSCFMLQQIEEPCSLGVHAAVVIPPTWILRARRPQNTLKASKKKKRASFKRKSSKKGPEEGRWRPFIIRPTPSPLMKPLLVFVNPKSGGNQGAKIIQSFLWYLNPRQVFDLSQGGPKEALEMYRKVHNLRILACGGDGTVGWILSTLDQLRLKPPPPVAILPLGTGNDLARTLNWGGGYTDEPVSKILSHVEEGNVVQLDRWDLHAEPNPEAGPEDRDEGATDRLPLDVFNNYFSLGFDAHVTLEFHESREANPEKFNSRFRNKMFYAGTAFSDFLMGSSKDLAKHIRVVCDGMDLTPKIQDLKPQCVVFLNIPRYCAGTMPWGHPGEHHDFEPQRHDDGYLEVIGFTMTSLAALQVGGHGERLTQCREVVLTTSKAIPVQVDGEPCKLAASRIRIALRNQATMVQKAKRRSAAPLHSDQQPVPEQLRIQVSRVSMHDYEALHYDKEQLKEASVPLGTVVVPGDSDLELCRAHIERLQQEPDGAGAKSPTCQKLSPKWCFLDATTASRFYRIDRAQEHLNYVTEIAQDEIYILDPELLGASARPDLPTPTSPLPTSPCSPTPRSLQGDAAPPQGEELIEAAKRNDFCKLQELHRAGGDLMHRDEQSRTLLHHAVSTGSKDVVRYLLDHAPPEILDAVEENGETCLHQAAALGQRTICHYIVEAGASLMKTDQQGDTPRQRAEKAQDTELAAYLENRQHYQMIQREDQETAV

SEQ ID NO: 40 - protein, 862 aa, Homo sapiens
MNIQEQGFPLDLGASFTEDAPRPPVPGEEGELVSTDPRPASYSFCSGKGVGIKGETSTATPRRSDLDLGYEPEGSASPTPPYLKWAESLHSLLDDQDGISLFRTFLKQEGCADLLDFWFACTGFRKLEPCDSNEEKRLKLARAIYRKYILDNNGIVSRQTKPATKSFIKGCIMKQLIDPAMFDQAQTEIQATMEENTYPSFLKSDIYLEYTRTGSESPKVCSDQSSGSGTGKGISGYLPTLNEDEEWKCDQDMDEDDGRDAAPPGRLPQKLLLETAAPRVSSSRRYSEGREFRYGSWREPVNPYYVNAGYALAPATSANDSEQQSLSSDADTLSLTDSSVDGIPPYRIRKQHRREMQESVQVNGRVPLPHIPRTYRVPKEVRVEPQKFAEELIHRLEAVQRTREAEEKLEERLKRVRMEEEGEDGDPSSGPPGPCHKLPPAPAWHHFPPRCVDMGCAGLRDAHEENPESILDEHVQRVLRTPGRQSPGPGHRSPDSGHVAKMPVALGGAASGHGKHVPKSGAKLDAAGLHHHRHVHHHVHHSTARPKEQVEAEATRRAQSSFAWGLEPHSHGARSRGYSESVGAAPNASDGLAHSGKVGVACKRNAKKAESGKSASTEVPGASEDAEKNQKIMQWIIEGEKEISRHRRTGHGSSGTRKPQPHENSRPLSLEHPWAGPQLRTSVQPSHLFIQDPTMPPHPAPNPLTQLEEARRRLEEEEKRASRAPSKQRYVQEVMRRGRACVRPACAPVLHVVPAVSDMELSETETRSQRKVGGGSAQPCDSIVVAYYFCGEPIPYRTLVRGRAVTLGQFKELLTKKGSYRYYFKKVSDEFDCGVVFEEVREDEAVLPVFEEKIIGKVEKVD

SEQ ID NO: 41 - protein, 322 aa, Homo sapiens
MEVANFYYEADCLAAAYGGKAAPAAPPAARPGPRPPAGELGSIGDHERAIDFSPYLEPLGAPQAPAPATATDTFEAAPPAPAPAPASSGQHHDFLSDLFSDDYGGKNCKKPAEYGYVSLGRLGAAKGALHPGCFAPLHPPPPPPPPPAELKAEPGFEPADCKRKEEAGAPGGGAGMAAGFPYALRAYLGYQAVPSGSSGSLSTSSSSSPPGTPSPADAKAPPTACYAGAAPAPSQVKSKAKKTVDKHSDEYKIRRERNNIAVRKSRDKAKMRNLETQHKVLELTAENERLQKKVEQLSRELSTLRNLFKQLPEPLLASSGHC

SEQ ID NO: 42 - protein, 673 aa, Homo sapiens
MDLTKMGMIQLQNPSHPTGLLCKANQMRLAGTLCDVVIMVDSQEFHAHRTVLACTSKMFEILFHRNSQHYTLDFLSPKTFQQILEYAYTATLQAKAEDLDDLLYAAEILEIEYLEEQCLKMLETIQASDDNDTEATMADGGAEEEEDRKARYLKNIFISKHSSEESGYASVAGQSLPGPMVDQSPSVSTSFGLSAMSPTKAAVDSLMTIGQSLLQGTLQPPAGPEEPTLAGGGRHPGVAEVKTEMMQVDEVPSQDSPGAAESSISGGMGDKVEERGKEGPGTPTRSSVITSARELHYGREESAEQVPPPAEAGQAPTGRPEHPAPPPEKHLGIYSVLPNHKADAVLSMPSSVTSGLHVQPALAVSMDFSTYGGLLPQGFIQRELFSKLGELAVGMKSESRTIGEQCSVCGVELPDNEAVEQHRKLHSGMKTYGCELCGKRFLDSLRLRMHLLAHSAGAKAFVCDQCGAQFSKEDALETHRQTHTGTDMAVFCLLCGKRFQAQSALQQHMEVHAGVRSYICSECNRTFPSHTALKRHLRSHTGDHPYECEFCGSCFRDESTLKSHKRIHTGEKPYECNGCGKKFSLKHQLETHYRVHTGEKPFECKLCHQRSRDYSAMIKHLRTHNGASPYQCTICTEYCPSLSSMQKHMKGHKPEEIPPDWRIEKTYLYLCYV

SEQ ID NO: 43 - protein, 383 aa, Homo sapiens
MGVKASQTGFVVLVLLQCCSAYKLVCYYTSWSQYREGDGSCFPDALDRFLCTHIIYSFANISNDHIDTWEWNDVTLYGMLNTLKNRNPNLKTLLSVGGWNFGSQRFSKIASNTQSRRTFIKSVPPFLRTHGFDGLDLAWLYPGRRDKQHFTTLIKEMKAEFIKEAQPGKKQLLLSAALSAGKVTIDSSYDIAKISQHLDFISIMTYDFHGAWRGTTGHHSPLFRGQEDASPDRFSNTDYAVGYMLRLGAPASKLVMGIPTFGRSFTLASSETGVGAPISGPGIPGRFTKEAGTLAYYEICDFLRGATVHRILGQQVPYATKGNQWVGYDDQESVKSKVQYLKDRQLAGAMVWALDLDDFQGSFCGQDLRFPLTNAIKDALAAT

SEQ ID NO: 44 - protein, 1548 aa, Homo sapiens
MLRARPEALMLLGALLTGSLGPSGNQDALSLPWEVQRYDGWFNNLRHHERGAVGCRLQRRVPANYADGVYQALEEPQLPNPRRLSNAATRGIAGLPSLHNRTVLGVFFGYHVLSDVVSVETPGCPAEFLNIRIPPGDPVFDPDQRGDVVLPFQRSRWDPETGRSPSNPRDLANQVTGWLDGSAIYGSSHSWSDALRSFSGGQLASGPDPAFPRDSQNPLLMWAAPDPATGQNGPRGLYAFGAERGNREPFLQALGLLWFRYHNLWAQRLARQHPDWEDEELFQHARKRVIATYQNIAVYEWLPSFLQKTLPEYTGYRPFLDPSISPEFVVASEQFFSTMVPPGVYMRNASCHFRKVLNKGFQSSQALRVCNNYWIRENPNLNSTQEVNELLLGMASQISELEDNIVVEDLRDYWPGPGKFSRTDYVASSIQRGRDMGLPSYSQALLAFGLDIPRNWSDLNPNVDPQVLEATAALYNQDLSQLELLLGGLLESHGDPGPLFSAIVLDQFVRLRDGDRYWFENTRNGLFSKKEIEDIRNTTLRDVLVAVINIDPSALQPNVFVWHKGAPCPQPKQLTTDGLPQCAPLTVLDFFEGSSPGFAITIIALCCLPLVSLLLSGVVAYFRGREHKKLQKKLKESVKKEAAKDGVPAMEWPGPKERSSPIIIQLLSDRCLQVLNRHLTVLRVVQLQPLQQVNLILSNNRGCRTLLLKIPKEYDLVLLFSSEEERGAFVQQLWDFCVRWALGLHVAEMSEKELFRKAVTKQQRERILEIFFRHLFAQVLDINQADAGTLPLDSSQKVREALTCELSRAEFAESLGLKPQDMFVESMFSLADKDGNGYLSFREFLDILVVFMKGSPEDKSRLMFTMYDLDENGFLSKDEFFTMMRSFIEISNNCLSKAQLAEVVESMFRESGFQDKEELTWEDFHFMLRDHDSELRFTQLCVKGGGGGGNGIRDIFKQNISCRVSFITRTPGERSHPQGLGPPAPEAPELGGPGLKKRFGKKAAVPTPRLYTEALQEKMQRGFLAQKLQQYKRFVENYRRHIVCVAIFSAICVGVFADRAYYYGFASPPSDIAQTTLVGIILSRGTAASVSFMFSYILLTMCRNLITFLRETFLNRYVPFDAAVDFHRWIAMAAVVLAILHSAGHAVNVYIFSVSPLSLLACIFPNVFVNDGSKLPQKFYWWFFQTVPGMTGVLLLLVLAIMYVFASHHFRRRSFRGFWLTHHLYILLYALLIIHGSYALIQLPTFHIYFLVPAIIYGGDKLVSLSRKKVEISVVKAELLPSGVTYLQFQRPQGFEYKSGQWVRIACLALGTTEYHPFTLTSAPHEDTLSLHIRAVGPWTTRLREIYSSPKGNGCAGYPKLYLDGPFGEGHQEWHKFEVSVLVGGGIGVTPFASILKDLVFKSSLGSQMLCKKIYFIWVTRTQRQFEWLADIIQEVEENDHQDLVSVHIYVTQLAEKFDLRTTMLYICERHFQKVLNRSLFTGLRSITHFGRPPFEPFFNSLQEVHPQVRKIGVFSCGPPGMTKNVEKACQLVNRQDRAHFMHHYENF

SEQ ID NO: 45 - protein, 146 aa, Homo sapiens
MAGPLRAPLLLLAILAVALAVSPAAGSSPGKPPRLVGGPMDASVEEEGVRRALDFAVGEYNKASNDMYHSRALQVVRARKQIVAGVNYFLDVELGRTTCTKTQPNLDNCPFHDQPHLKRKAFCSFQIYAVPWQGTMTLSKSTCQDA

(19) United States
(12) Patent Application Publication
(10) Pub. No.: US 2026/0237515 A1
(43) Pub. Date: Aug. 13, 2026

(54) AQUEOUS HUMOR PROTEOME AND METASTATIC MELANOMA

(71) Applicant: The Saban Research Institute; Children's Hospital Los Angeles, Los Angeles, CA (US)

(72) Inventors: Jesse Berry, Manhattan Beach, CA (US); Liya Xu, San Diego, CA (US); Chen-Ching Peng, Los Angeles, CA (US)

(21) Appl. No.: 19/160,710

(22) PCT Filed: Feb. 28, 2024
(86) PCT No.: PCT/US2024/017765

§ 371 Date: Aug. 28, 2025

Related U.S. Application Data
(60) Provisional application No. 63/448,838, filed on Feb. 28, 2023.

(51) Int. Cl. G16H 50/30; G01N 33/575; G16H 50/20

(52) U.S. Cl. CPC G16H 50/30; G01N 33/57557; G01N 33/57585; G16H 50/20; G01N 2570/00; G01N 2800/56; G01N 2800/60

(57) ABSTRACT

Provided herein is a method to stratify risk of metastasis in a uveal melanoma (UM) patient to class 1 (low metastatic risk) or class 2 (high metastatic risk) comprising providing an aqueous humor (AH) liquid biopsy sample from said patient and determining modulation of expression of one or more genes in said sample.

### PRIORITY

This application claims the benefit of U.S. Provisional Application No. 63/448,838, filed Feb. 28, 2023, the content of which is herein incorporated by reference in its entirety.

### BACKGROUND

Uveal melanoma (UM) is the most common primary intraocular cancer, characterized by tumors arising from the choroid, iris, and ciliary body (1). Globe conserving treatment most commonly entails plaque brachytherapy or proton beam radiotherapy, with enucleation reserved for very large tumors. Even after treatment of the primary tumor, approximately half of all patients with UM will develop metastatic disease, resulting in the need for metastatic surveillance of these patients (2). However, as half of these patients may not develop metastatic cancer, stratifying risk of metastasis in these patients can lead to more patient-specific metastatic surveillance recommendations.

### SUMMARY

One aspect provides a method to stratify risk of metastasis in a uveal melanoma (UM) patient to class 1 (low metastatic risk) or class 2 (high metastatic risk) comprising providing an aqueous humor (AH) liquid biopsy sample from said patient and determining modulation of expression of one or more genes in said sample, wherein said modulation of expression of said one or more genes is correlated with class 1 or class 2.

Another aspect provides a method to determine an increased risk or likelihood of metastasis in a uveal melanoma (UM) patient comprising providing an aqueous humor (AH) liquid biopsy sample from said patient and determining modulation of expression of one or more genes in said sample, wherein said modulation of expression of said one or more genes is correlated with class 2, high metastatic risk.

One aspect provides a method to stratify risk of metastasis in a uveal melanoma (UM) patient to class 1 (low metastatic risk) or class 2 (high metastatic risk) comprising providing an aqueous humor (AH) liquid biopsy sample from said patient and determining modulation of expression of one or more genes in said sample, wherein said modulation of expression of said one or more genes is correlated with class 1 (low metastatic risk) or class 2 (high metastatic risk).

In some aspects, the said one or more genes are NUCB2, DBI, CSTB, CD14, SCG2, GNLY, RGMB, CLPS, CXCL10, TGFBR3, IGFBP3, CTSF, VWA1, SERPINE1, HGF, IL18BP, MSR1, TREM2, TNFRSF1B, PLAUR, OSCAR, PILRA, TNFRSF11A, ANGPTL4, SMAD5, TNFRSF12A, SMOC2, TNFRSF1A, ST6GAL1, LAIR1, CD74, DPP7, MAPT, TDGF1, PMVK, CNTN2, AMN, SLC39A5, DGKZ, AXIN1, CEBPB, ZBTB16, CHI3L1, DUOX2 or CST3. In one aspect, one or more of NUCB2, DBI, CSTB, CD14, SCG2, GNLY, RGMB, CLPS, CXCL10, TGFBR3, IGFBP3, CTSF, VWA1, SERPINE1, HGF, IL18BP, MSR1, TREM2, TNFRSF1B, PLAUR, OSCAR, PILRA, TNFRSF11A, ANGPTL4, SMAD5, TNFRSF12A, SMOC2, TNFRSF1A, ST6GAL1, LAIR1 or CD74 genes are upregulated for class 2. In one aspect, one or more of DPP7, MAPT, TDGF1, PMVK, CNTN2, AMN, SLC39A5, DGKZ, AXIN1, CEBPB, ZBTB16, CHI3L1, DUOX2 or CST3 genes are downregulated for class 2. In aspect, the one or more genes are human genes.

In some embodiments, UM tumors present in said subject are too small to provide biopsied tissue. One aspect further comprises treating said subject for UM and/or UM metastases, wherein the treatment is surgery (e.g., resection, enucleation, exenteration), radiation (e.g., external radiation (e.g., charged-particle external-beam radiation, gamma knife) and/or internal radiation (e.g., localized plaque radiation), photocoagulation, chemotherapy, immunotherapy and/or thermotherapy.

### BRIEF DESCRIPTION OF THE DRAWINGS

FIG. 1 Patient demographic. GEP, gene expression profile; Dx, diagnosis; PRAME, preferentially expressed antigen in melanoma; AJCC, American Joint Committee on Cancer (AJCC).

FIGS. 2A-2B Normalized protein expression (NPX) distribution from 20 UM AH samples. (A) protein count versus NPX distribution and (B) principle component analysis of 1469 proteins in 20 UM AH.

FIGS. 3A-3B Differentially expressed AH proteins in GEP2 versus GEP1 samples. (A) Differentially expressed AH proteins (DEPs) in GEP2 versus GEP1 samples are defined as −log (P) greater than 2 (p value<0.01) and log 2 (fold change) is greater than 1 (fold-change>2 or <0.5, GEP2 over GEP1). Red dots denote 31 upregulated DEPs (uDEPs) and blue dots are 14 downregulated DEPs (dDEPs). The P value was calculated by the Mann-Whitney U test. (B) Unsupervised clustering of 20 UM AH samples using 45 DEPs. GEP classes, PRAME status, AJCC stages and the color chart representing expression levels for each protein are indicated.

FIGS. 4A-4E Biological relevance of the DEPs. (A) Qiagen Ingenuity Pathway Analysis (IPA) function annotation analysis of the 45 DEPs. Z-score, −log p values and number of DEPs (yellow circles) contributing to each pathway are indicated. (B) Upstream regulator analysis of the 45 DEPs. Top 5 significant upstream regulators are plotted. Increased (red) and decreased (blue) z-score and −log p values are shown. (C) Protein expression levels of potential upstream regulators, TNF, FGF2, IL-1 receptor 1 and 2 and SPRY2 in PEA platform. (D) Potential downstream targets of IL-1 receptor and SPRY2 in 45 DEPs. Color of circles represent number of cellular pathways the targets involved in. (E) Protein expression levels of IL-1 receptor and SPRY2 targets, HGF, SERPINE1, TNFRSF1B. IGFBP3, PLAUR, ANGPTL4, CXCL10 and ST6GAL1. P values were calculated by the Mann-Whitney U test.

FIG. A1 Principle component analysis of normalized protein expression (NPX) distribution from 3 CAT, 4 GLC and 20 UM AH samples. CAT, congenital cataract; GLC, congenital glaucoma.

FIG. A2 Somatic copy number alterations in UM_005 AH samples. Gains and losses of chromosomes are shown.

### DETAILED DESCRIPTION OF THE INVENTION

Gene expression profiling (GEP) is clinically validated to stratify risk of metastasis by assigning uveal melanoma (UM) patients to two highly prognostic molecular classes: class 1 (low metastatic risk) and class 2 (high metastatic risk). However, GEP requires intraocular tumor biopsy which is limited by small tumor size and tumor heterogeneity; furthermore, there are risks of retinal hemorrhage, bleeding, or tumor dissemination. Thus, ocular liquid biopsy has emerged as a less invasive alternative. Provided herein is the aqueous humor (AH) proteome related to the advanced GEP class 2 using diagnostic AH liquid biopsy specimens. Twenty AH samples were collected from patients with UM, grouped by GEP classes. Protein expression levels of 1472 targets were analyzed, compared between GEP classes, and correlated with clinical features. Significant differentially expressed proteins (DEPs) were subjected to analysis for cellular pathway and upstream regulator identification. Results showed that 45 DEPs detected in the AH could differentiate GEP class 1 and 2 at diagnosis. IL1R and SPRY2 are potential upstream regulators for the 8/45 DEPs that contribute to metastasis-related pathways. AH liquid biopsy provides a method to determine metastatic potential for patients in the absence of tumor biopsy.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14th Edition, by R. J. Lewis, John Wiley & Sons, New York, N.Y., 2001.

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is di-substituted.

As used herein, “or” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.”

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the endpoints of a recited range as discuss above in this paragraph.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group.

Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

As used herein, the term “comparing” refers to making an assessment of how the proportion, level or cellular localization of one or more biomarkers in a sample from a patient relates to the proportion, level or cellular localization of the corresponding one or more biomarkers in a standard or control sample. For example, “comparing” may refer to assessing whether the proportion, level, or cellular localization of one or more biomarkers in a sample from a patient is the same as, more or less than, or different from the proportion, level, or cellular localization of the corresponding one or more biomarkers in a standard or control sample or another patient sample. More specifically, the term may refer to assessing whether the proportion, level, or cellular localization of one or more biomarkers in a sample from a patient is the same as, more or less than, different from or otherwise corresponds (or not) to the proportion, level, or cellular localization of predefined biomarker levels that correspond to, for example, a patient UM.

As used herein, the terms “indicates” or “correlates” (or “indicating” or “correlating,” or “indication” or “correlation,” depending on the context) in reference to a parameter, e.g., a modulated proportion, level, or cellular localization in a sample from a patient, may mean that the patient has or at risk of having metastatic cancer.

The terms “patient,” “individual,” or “subject” are used interchangeably herein, and refer to a mammal, particularly, a human. As used herein, a “subject in need thereof” is a patient, animal, mammal, or human, who will benefit from the method of this invention. The patient may have mild, intermediate or severe disease, or may only experience transient, or fluctuating symptoms. The patient may be an individual, at risk of developing a disease, in need of treatment or in need of diagnosis based on particular symptoms or family history. In some cases, the terms may refer to treatment of animals, in such as veterinary applications (e.g., companion animals (e.g., cats or dogs) or livestock (e.g., pigs, cows, horses)).

The terms “measuring” and “determining” are used interchangeably throughout and refer to methods which include obtaining a patient sample and/or detecting the level of a biomarker(s) in a sample. In one embodiment, the terms refer to obtaining a patient sample and detecting the level of one or more biomarkers in the sample. In another embodiment, the terms “measuring” and “determining” mean detecting level of one or more biomarkers in a patient sample. Measuring can be accomplished by methods known in the art and those further described herein including. The term “measuring” is also used interchangeably throughout with the term “detecting.”

The terms “sample,” “patient sample,” “biological sample,” and the like, encompass a variety of sample types obtained from a patient, individual, or subject and can be used in a diagnostic or monitoring assay. The patient sample may be obtained from a healthy subject, a diseased patient or a patient having associated symptoms. Moreover, a sample obtained from a patient can be divided and only a portion may be used for diagnosis. Further, the sample, or a portion thereof, can be stored under conditions to maintain sample for later analysis.

Various methodologies of the instant invention include a step that involves comparing a value, level, feature, characteristic, property, etc. to a “suitable control,” referred to interchangeably herein as an “appropriate control” or a “control sample.” A “suitable control,” “appropriate control” or a “control sample” is any control or standard familiar to one of ordinary skill in the art useful for comparison purposes. In one embodiment, a “suitable control” or “appropriate control” is a value, level, feature, characteristic, property, etc., determined in a cell, organ, or patient, e.g., a control or normal cell, organ, or patient, exhibiting, for example, normal traits. For example, the biomarkers of the present invention may be assayed in a sample from an unaffected individual (UI) or a normal control individual (NC) (both terms are used interchangeably herein) or other affected individual. In another embodiment, a “suitable control” or “appropriate control” is a value, level, feature, characteristic, property, etc. determined prior to performing a therapy on a patient. In yet another embodiment, a transcription rate, mRNA level, translation rate, protein level, biological activity, cellular characteristic or property, genotype, phenotype, etc. can be determined prior to, during, or after administering a therapy into a cell, organ, or patient. In a further embodiment, a “suitable control” or “appropriate control” is a predefined value, level, feature, characteristic, property, etc.

The term “otherwise identical sample,” as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art, especially in light of the detailed disclosure provided herein. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

The terms “treating,” “treat” and “treatment” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

As used herein, the term “pharmaceutically-acceptable carrier” means a chemical composition with which an appropriate compound or derivative can be combined and which, following the combination, can be used to administer the appropriate compound to a subject. “Pharmaceutically acceptable” means physiologically tolerable, for either human or veterinary application. As used herein, “pharmaceutical compositions” include formulations for human and veterinary use.

Methods involving conventional molecular biology techniques are described herein. Such techniques are generally known in the art and are described in detail in methodology treatises, such as Molecular Cloning: A Laboratory Manual, Sambrook et al., Cold Spring Harbor Laboratory Press, Cold Spring Harbor, N.Y.

### Genes/Biomarkers of the Invention

The biomarkers of the present invention can be used in diagnostic tests to assess, determine, confirm and/or qualify (used interchangeably herein) metastasis in a UM patient. NUCB2, DBI, CSTB, CD14, SCG2, GNLY, RGMB, CLPS, CXCL10, TGFBR3, IGFBP3, CTSF, VWA1, SERPINE1, HGF, IL18BP, MSR1, TREM2, TNFRSF1B, PLAUR, OSCAR, PILRA, TNFRSF11A, ANGPTL4, SMAD5, TNFRSF12A, SMOC2, TNFRSF1A, ST6GAL1, LAIR1, CD74, DPP7, MAPT, TDGF1, PMVK, CNTN2, AMN, SLC39A5, DGKZ, AXIN1, CEBPB, ZBTB16, CHI3L1, DUOX2 or CST3.

For example, one or more of NUCB2, DBI, CSTB, CD14, SCG2, GNLY, RGMB, CLPS, CXCL10, TGFBR3, IGFBP3, CTSF, VWA1, SERPINE1, HGF, IL18BP, MSR1, TREM2, TNFRSF1B, PLAUR, OSCAR, PILRA, TNFRSF11A, ANGPTL4, SMAD5, TNFRSF12A, SMOC2, TNFRSF1A, ST6GAL1, LAIR1 or CD74 genes are upregulated (increased RNA and/or protein expression) for class 2. In another example, one or more of DPP7, MAPT, TDGF1, PMVK, CNTN2, AMN, SLC39A5, DGKZ, AXIN1, CEBPB, ZBTB16, CHI3L1, DUOX2 or CST3 genes are downregulated (decreased RNA and/or protein expression) for class 2.

Examples of human proteins are as follows:

| NUCB2 (nucleobindin-2; NP_001317156; NP_005004; NP_001339590; NP_001339591; | |
|---|---|
| NP_001339592) | |
| (SEQ ID NO: 1) | |
| 1 | mrwrtillqy cfllitcllt aleavpidid ktkvqnihpv esakieppdt glyydeylkq |
| 61 | vidvletdkh freklqkadi eeiksgrlsk eldlvshhvr tkldelkrqe vgrlrmlika |
| 121 | kldslqdigm dhqallkqfd hlnhlnpdkf estdldmlik aatsdlehyd ktrheefkky |
| 181 | emmkeherre ylktlneekr keeeskfeem kkkhenhpkv nhpgskdqlk evweetdgld |
| 241 | pndfdpktff klhdvnsdgf ldeqelealf tkelekvydp kneeddmvem eeerlrmreh |
| 301 | vmnetldqqq ffteeelkey eniialqene lkkkadelqk qkeelqrqhd qleaqkleyh |
| 361 | qviqqmeqkk lqqgippsgp agelkfephi |
| DBI (diazepam binding inhibitor; NP_001073331; NP_001073332; NP_001171488; | |
| NP_001171512NP_001171513) | |
| (SEQ ID NO: 2) | |
| 1 | msqaefekaa eevrhlktkp sdeemlfiyg hykqatvgdi nterpgmldf tgkakwdawn |
| 61 | elkgtskeda mkayinkvee lkkkygi |
| CSTB (Cystatin-B; NP_000091) | |
| (SEQ ID NO: 3) | |
| 1 | mmcgapsatq pataetqhia dqvrsqleek enkkfpvfka vsfksqvvag tnyfikvhvg |
| 61 | dedfvhlrvf qslphenkpl tlsnyqtnka khdeltyf |
| CD14 (cluster of differentiation 14; NP_000582; NP_001035110; NP_001167575; | |
| NP_001167576) | |
| (SEQ ID NO: 4) | |
| 1 | merascllll llplvhvsat tpepceldde dfrcvcnfse pqpdwseafq cvsaveveih |
| 61 | agglnlepfl krvdadadpr qyadtvkalr vrrltvgaaq vpaqllvgal rvlaysrlke |
| 121 | ltledlkitg tmpplpleat glalsslrlr nvswatgrsw laelqqwlkp glkvlsiaqa |
| 181 | hspafsceqv rafpaltsld lsdnpglger glmaalcphk fpaiqnlalr ntgmetptgv |
| 241 | caalaaagvq phsldlshns lratvnpsap rcmwssalns lnlsfagleq vpkglpaklr |
| 301 | vldlscnrln rapqpdelpe vdnltldgnp flvpgtalph egsmnsgvvp acarstlsvg |
| 361 | vsgtlvllqg argfa |
| SCG2 (secretogranin II (chromogranin C); NP_003460) | |
| (SEQ ID NO: 5) | |
| 1 | maeakthwlg aalsliplif lisgaeaasf qrnqllqkep dlrlenvqkf pspemirale |
| 61 | yienlrqqah keesspdynp yqgvsvplqq kengdeshlp erdslseedw mriilealrq |
| 121 | aenepqsapk enkpyalnse knfpmdmsdd yetqqwperk lkhmqfppmy eensrdnpfk |
| 181 | rtneiveeqy tpqslatles vfqelgkltg pnnqkrermd eeqklytdde ddiykannia |
| 241 | yedvvggedw npveekiesq tqeevrdske niekneqind emkrsgqlgi qeedlrkesk |
| 301 | dqlsddvskv iaylkrlvna agsgrlqngq ngeratrlfe kpldsqsiyq lieisrnlqi |
| 361 | ppedliemlk tgekpngsve pereldlpvd lddiseadld hpdlfqnrml sksgypktpg |
| 421 | ragtealpdg lsvedilnll gmesaanqkt syfpnpynqe kvlprlpyga grsrsnqlpk |
| 481 | aawiphvenr qmayenlndk dqelgeylar mlvkypeiin snqvkrvpgq gsseddlqee |
| 541 | eqieqaikeh lnqgssqetd klapvskrfp vgppknddtp nrqywdedll mkvleylnqe |
| 601 | kaekgrehia kramenm |
| GNLY (Granulysin; NP_001289687; NP_006424; NP_036615 | |
| (SEQ ID NO: 6) | |
| 1 | matwalllla amllgnpgle vsvspkgknt sgresgfgwa iwmeglvfsr lspeyydlar |
| 61 | ahlrdeeksc pclaqegpqg dlltktqelg rdyrtcltiv qklkkmvdkp tqrsvsnaat |
| 121 | rvcrtgrsrw rdvcrnfmrr yqsrvtqglv agetaqqice dlrlcipstg pl |
| RGMB (Repulsive guidance molecule B; NP_001012779; NP_001353437; NP_001353438; | |
| NP_001353439; NP_001353440) | |
| (SEQ ID NO: 7) | |
| 1 | mirkkrkrsa ppgpcrshgp rpatapappp speptrpawt gmglraapss aaaaaaeveq |
| 61 | rrspglcppp lellllllfs lgllhagdcq qpaqcriqkc ttdfvsltsh lnsavdgfds |
| 121 | efckalraya gctqrtskac rgnlvyhsav lgisdlmsqr ncskdgptss tnpevthdpc |
| 181 | nyhshagare hrrgdqnpps ylfcglfgdp hlrtfkdnfq tckvegawpl idnnylsvqv |
| 241 | tnvpvvpgss atatnkitii fkahhectdq kvyqavtddl paafvdgtts ggdsdakslr |
| 301 | iveresghyv emharyigtt vfvrqvgryl tlairmpedl amsyeesqdl qlcvngcpls |
| 361 | eriddgqgqv sailghslpr tslvqawpgy tletantqch ekmpvkdiyf qscvfdlltt |
| 421 | gdanftaaah saledvealh prkerwhifp ssgngtprgg sdlsvslglt clilivfl |
| CLPS (colipase; NP_001239526; NP_001239527; NP_001823) | |
| (SEQ ID NO: 8) | |
| 1 | millpqppkk lglqengelc mnsaqcksnc cqhssalgla rctsmasens ecsvktlygi |
| 61 | yykcpcergl tcegdktivg sitntnfgic hdagrskq |
| CXCL10 (C-X-C motif chemokine ligand 10; NP_001556) | |
| (SEQ ID NO: 9) | |
| 1 | mnqtailicc lifltlsgiq gvplsrtvrc tcisisnqpv nprsleklei ipasqfcprv |
| 61 | eiiatmkkkg ekrclnpesk aiknllkavs kerskrsp |
| TGFBR3 (Betaglycan also known as Transforming growth factor beta receptor III; | |
| NP_001182612; NP_001182613; NP_003234) | |
| (SEQ ID NO: 10) | |
| 1 | mtshyviaif almssclata gpepgalcel spvsashpvq almesftvls gcasrgttgl |
| 61 | pqevhvlnlr tagqgpgqlq revtlhlnpi ssvhihhksv vfllnsphpl vwhlkterla |
| 121 | tgvsrlflvs egsvvqfssa nfsltaetee rnfphgnehl lnwarkeyga vtsftelkia |
| 181 | rniyikvged qvfppkcnig knflslnyla eylqpkaaeg cvmssqpqne evhiielitp |
| 241 | nsnpysafqv ditidirpsq edlevvknli lilkckksvn wviksfdvkg slkiiapnsi |
| 301 | gfgkesersm tmtksirddi pstqgnlvkw aldngyspit sytmapvanr fhlrlennee |
| 361 | mgdeevhtip pelrilldpg alpalqnppi rggegqnggl pfpfpdisrr vwneegedgl |
| 421 | prpkdpvips iqlfpglrep eevqgsvdia lsvkcdnekm ivavekdsfq asgysgmdvt |
| 481 | lldptckakm ngthfvlesp lngcgtrprw saldgvvyyn siviqvpalg dssgwpdgye |
| 541 | dlesgdngfp gdmdegdasl ftrpeivvfn cslqqvrnps sfqeqphgni tfnmelyntd |
| 601 | lflvpsqgvf svpenghvyv evsvtkaeqe lgfaiqtcfi spysnpdrms hytiienicp |
| 661 | kdesvkfysp krvhfpipqa dmdkkrfsfv fkpvfntsll flqceltlct kmekhpqklp |
| 721 | kcvppdeact sldasiiwam mqnkktftkp lavihheaes kekgpsmkep npisppifhg |
| 781 | ldtltvmgia faafvigall tgalwyiysh tgetagrqqv ptsppasens saahsigstq |
| 841 | stpcssssta |
| IGFBP3 (Insulin-like growth factor-binding protein 3; NP_000589; NP_001013416) | |
| (SEQ ID NO: 11) | |
| 1 | mqrarptlwa aaltllvllr gppvaragas saglgpvvrc epcdaralaq capppavcae |
| 61 | lvrepgcgcc ltcalsegqp cgiytercgs glrcqpspde arplqalldg rglcvnasav |
| 121 | srlrayllpa ppapgnases eedrsagsve spsvssthrv sdpkfhplhs kiiiikkgha |
| 181 | kdsqrykvdy esqstdtqnf sseskretey gpcrremedt lnhlkflnvl sprgvhipnc |
| 241 | dkkgfykkkq crpskgrkrg fcwcvdkygq plpgyttkgk edvhcysmqs k |
| CTSF (Cathepsin F; NP_003784) | |
| (SEQ ID NO: 12) | |
| 1 | mapwlqllsl lgllpgavaa paqpraasfq awgppspell aptrfalemf nrgraagtra |
| 61 | vlglvrgrvr ragqgslysl eatleeppcn dpmvcrlpvs kktllcsfqv ldelgrhvll |
| 121 | rkdcgpvdtk vpgagepksa ftqgsamiss lsqnhpdnrn etfssvisll nedplsqdlp |
| 181 | vkmasifknf vitynrtyes keearwrlsv fvnnmvraqk iqaldrgtaq ygvtkfsdlt |
| 241 | eeefrtiyln tllrkepgnk mkqaksvgdl appewdwrsk gavtkvkdqg mcgscwafsv |
| 301 | tgnvegqwfl nqgtllslse qelldcdkmd kacmgglpsn aysaiknlgg leteddysyq |
| 361 | ghmqscnfsa ekakvyinds velsqneqkl aawlakrgpi svainafgmq fyrhgisrpl |
| 421 | rplcspwlid havllvgygn rsdvpfwaik nswgtdwgek gyyylhrgsg acgvntmass |
| 481 | avvd |
| VWA1 (von Willebrand factor A domain containing 1; NM_022834.5 → NP_073745.2) | |
| (SEQ ID NO: 13) | |
| 1 | mlpwtalgla lslrlalars gaergppasa prgdlmflld ssasvshyef srvrefvgql |
| 61 | vaplplgtga lraslvhvgs rpytefpfgq hssgeaaqda vrasaqrmgd thtglalvya |
| 121 | keqlfaeasg arpgvpkvlv wvtdggssdp vgppmqelkd lgvtvfivst grgnflelsa |
| 181 | aasapaekhl hfvdvddlhi ivqelrgsil damrpqqlha teitssgfrl awpplltads |
| 241 | gyyvlelvps aqpgaarrqq lpgnatdwiw agldpdtdyd valvpesnvr llrpqilrvr |
| 301 | trpgeagpga sgpesgagpa ptqlaalpap eeagperivi sharprslrv swapalgsaa |
| 361 | algyhvqfgp lrggeaqrve vpagrncttl qglapgtayl vtvtaafrsg resalsakac |
| 421 | tpdgprprpr pvpraptpgt asrep |
| SERPINE1 (Plasminogen activator inhibitor-1 (PAI-1) also known as endothelial | |
| plasminogen activator inhibitor (serpin E1; NP_000593; NP_000593.1) | |
| (SEQ ID NO: 14) | |
| 1 | mqmspaltcl vlglalvfge gsavhhppsy vahlasdfgv rvfqqvaqas kdrnvvfspy |
| 61 | gvasvlamlq lttggetqqq iqaamgfkid dkgmapalrh lykelmgpwn kdeisttdai |
| 121 | fvqrdlklvq gfmphffrlf rstvkqvdfs everarfiin dwvkthtkgm isnllgkgav |
| 181 | dqltrlvlvn alyfngqwkt pfpdssthrr lfhksdgstv svpmmaqtnk fnytefttpd |
| 241 | ghyydilelp yhgdtlsmfi aapyekevpl saltnilsaq lishwkgnmt rlprllvlpk |
| 301 | fsletevdlr kplenlgmtd mfrqfqadft slsdqeplhv aqalqkvkie vnesgtvass |
| 361 | stavivsarm apeeiimdrp flfvvrhnpt gtvlfmgqvm ep |
| HGF (Hepatocyte growth factor; NP_000592; NP_001010931; NP_001010932; | |
| NP_001010933; NP_001010934) | |
| (SEQ ID NO: 15) | |
| 1 | mwvtkllpal llqhvllhll llpiaipyae gqrkrrntih efkksakttl ikidpalkik |
| 61 | tkkvntadqc anrctrnkgl pftckafvfd karkqclwfp fnsmssgvkk efghefdlye |
| 121 | nkdyirncii gkgrsykgtv sitksgikcq pwssmipheh sflpssyrgk dlqenycrnp |
| 181 | rgeeggpwcf tsnpevryev cdipqcseve cmtcngesyr glmdhtesgk icqrwdhqtp |
| 241 | hrhkflpery pdkgfddnyc rnpdgqprpw cytldphtrw eycaiktcad ntmndtdvpl |
| 301 | etteciqgqg egyrgtvnti wngipcqrwd sqyphehdmt penfkckdlr enycrnpdgs |
| 361 | espwcfttdp nirvgycsqi pncdmshgqd cyrgngknym gnlsqtrsgl tcsmwdknme |
| 421 | dlhrhifwep dasklnenyc rnpdddahgp wcytgnplip wdycpisrce gdttptivnl |
| 481 | dhpviscakt kqlrvvngip trtnigwmvs lryrnkhicg gslikeswvl tarqcfpsrd |
| 541 | lkdyeawlgi hdvhgrgdek ckqvlnvsql vygpegsdlv lmklarpavl ddfvstidlp |
| 601 | nygctipekt scsvygwgyt glinydgllr vahlyimgne kcsqhhrgkv tlneseicag |
| 661 | aekigsgpce gdyggplvce qhkmrmvlgv ivpgrgcaip nrpgifvrva yyakwihkii |
| 721 | ltykvpqs |
| IL18BP (Interleukin-18-binding protein; NP_001034748; NP_001034749; | |
| NP_001138527; NP_001138529; NP_005690) | |
| (SEQ ID NO: 16) | |
| 1 | mtmrhnwtpd lsplwvlllc ahvvtllvra tpvsqtttaa tasvrstkdp cpsqppvfpa |
| 61 | akqcpalevt wpevevplng tlslscvacs rfpnfsilyw lgngsfiehl pgrlwegsts |
| 121 | rergstgtql ckalvleqlt palhstnfsc vlvdpeqvvq rhvvlaqlwa glratlpptq |
| 181 | ealpsshssp qqqg |
| MSR1 (Macrophage scavenger receptor 1; NP_002436; NP_619729; NP_619730; | |
| NP_001350673) | |
| (SEQ ID NO: 17) | |
| 1 | meqwdhfhnq qedtdscses vkfdarsmta llppnpknsp slqeklksfk aalialyllv |
| 61 | favlipligi vaaqllkwet kncsvsstna nditqsltgk gndseeemrf qevfmehmsn |
| 121 | mekriqhild meanlmdteh fqnfsmttdq rfndillqls tlfssvqghg naideisksl |
| 181 | islnttlldl qlnienlngk iqentfkqqe eiskleervy nvsaeimamk eeqvhleqei |
| 241 | kgevkvlnni tndlrlkdwe hsqtlrnitl iqgppgppge kgdrgptges gprgfpgpig |
| 301 | ppglkgdrga igfpgsrglp gyagrpgnsg pkgqkgekgs gntlrpvqlt dhiragps |
| TREM2 (Triggering receptor expressed on myeloid cells 2; NP_001258750; | |
| NP_061838) | |
| (SEQ ID NO: 18) | |
| 1 | meplrllill fvtelsgahn ttvfqgvagq slqvscpyds mkhwgrrkaw crqlgekgpc |
| 61 | qrvvsthnlw llsflrrwng staitddtlg gtltitlrnl qphdaglyqc qslhgseadt |
| 121 | lrkvlvevla dpldhrdagd lwfpgesesf edahvehsis raerhvkedd grkspgevpp |
| 181 | gtspacilat wppgllvllw qettlpehcf swtleagtg |
| TNFRSF1B (Tumor necrosis factor receptor 2 (TNFR2), also known as tumor | |
| necrosis factor receptor superfamily member 1B (TNFRSF1B); NP_001057) | |
| (SEQ ID NO: 19) | |
| 1 | mapvavwaal avglelwaaa halpaqvaft pyapepgstc rlreyydqta qmccskcspg |
| 61 | qhakvfctkt sdtvcdsced stytqlwnwv peclscgsrc ssdqvetqac treqnrictc |
| 121 | rpgwycalsk qegcrlcapl rkcrpgfgva rpgtetsdvv ckpcapgtfs nttsstdicr |
| 181 | phqicnvvai pgnasmdavc tstsptrsma pgavhlpqpv strsqhtqpt pepstapsts |
| 241 | fllpmgpspp aegstgdfal pvglivgvta lglliigvvn cvimtqvkkk plclqreakv |
| 301 | phlpadkarg tqgpeqqhll itapssssss lessasaldr raptrnqpqa pgveasgage |
| 361 | arastgssds spgghgtqvn vtcivnvcss sdhssqcssq asstmgdtds spsespkdeq |
| 421 | vpfskeecaf rsqletpetl lgsteekplp lgvpdagmkp s |
| PLAUR (Urokinase receptor, also known as urokinase plasminogen activator | |
| surface receptor (uPAR) or CD87 (Cluster of Differentiation 87), is a | |
| protein encoded in humans by the PLAUR gene; NP_001005376; NP_001005377; | |
| NP_001287966; NP_002650) | |
| (SEQ ID NO: 20) | |
| 1 | mghppllpll lllhtcvpas wglrcmqckt ngdcrveeca lgqdlcrtti vrlweegeel |
| 61 | elvekscths ektnrtlsyr tglkitslte vvcgldlcnq gnsgravtys rsrylecisc |
| 121 | gssdmscerg rhqslqcrsp eeqcldvvth wiqegeegrp kddrhlrgcg ylpgcpgsng |
| 181 | fhnndtfhfl kccnttkcne gpilelenlp qngrqcysck gnsthgcsse etflidcrgp |
| 241 | mnqclvatgt herslwgswl pcksttalrp pcceeaqath v |
| OSCAR (Osteoclast-associated immunoglobulin-like receptor; NP_001269278; | |
| NP_001269279 NP_570127; NP_573398; NP_573399) | |
| (SEQ ID NO: 21) | |
| 1 | malvlilqll tlwplchtdi tpsvppasyh pkpwlgaqpa tvvtpgvnvt lrcrapqpaw |
| 61 | rfglfkpgei apllfrdvss elaeffleev tpaqggsyrc cyrrpdwgpg vwsqpsdvle |
| 121 | llvteelprp slvalpgpvv gpganvslrc agrlrnmsfv lyregvaapl qyrhsaqpwa |
| 181 | dftllgarap gtyscyyhtp sapyvlsqrs evlviswege gpearpassa pgmqapgppp |
| 241 | sdpgaqapsl ssfrprglvl qpllpqtqds wdpapppsdp gv |
| PILRA (Paired immunoglobin like type 2 receptor alpha; NP_038467; NP_840056; | |
| NP_840057) | |
| (SEQ ID NO: 22) | |
| 1 | mgrplllpll plllppaflq psgstgsgps ylygvtqpkh lsasmggsve ipfsfyypwe |
| 61 | latapdvris wrrghfhrqs fystrppsih kdyvnrlfln wtegqksgfl risnlqkqdq |
| 121 | svyfcrveld trssgrqqwq siegtklsit qavttttqrp ssmtttwrls stttttglrv |
| 181 | tqgkrrsdsw hisletavgv avavtvlgim ilglicllrw rrrkgqqrtk attparepfq |
| 241 | nteepyenir negqntdpkl npkddgivya slalssstsp rappshrplk spqnetlysv |
| 301 | lka |
| TNFRSF11A (Receptor activator of nuclear factor κ B (RANK), also known | |
| as TRANCE receptor or TNFRSF11A; NP_001257878; NP_001257879; NP_001257880; | |
| NP_001265197; NP_003830) | |
| (SEQ ID NO: 23) | |
| 1 | maprarrrrp lfallllcal larlqvalqi appctsekhy ehlgrccnkc epgkymsskc |
| 61 | tttsdsvclp cgpdeyldsw needkcllhk vcdtgkalva vvagnsttpr rcactagyhw |
| 121 | sqdceccrrn tecapglgaq hplqlnkdtv ckpclagyfs dafsstdkcr pwtnctflgk |
| 181 | rvehhgteks davcssslpa rkppnephvy lpgliilllf asvalvaaii fgvcyrkkgk |
| 241 | altanlwhwi neacgrlsgd kem |
| ANGPTL4 (Angiopoietin-like 4; NP_001034756; NP_647475) | |
| (SEQ ID NO: 24) | |
| 1 | msgaptagaa lmlcaatavl lsaqggpvqs ksprfaswde mnvlahgllq lgqglrehae |
| 61 | rtrsqlsale rrlsacgsac qgtegstdlp lapesrvdpe vlhslqtqlk aqnsriqqlf |
| 121 | hkvaqqqrhl ekqhlriqhl qsqfglldhk hldhevakpa rrkrlpemaq pvdpahnvsr |
| 181 | lhhggwtviq rrhdgsvdfn rpweaykagf gdphgefwlg lekvhsitgd rnsrlavqlr |
| 241 | dwdgnaellq fsvhlggedt ayslqltapv agqlgattvp psglsvpfst wdqdhdlrrd |
| 301 | kncakslsgg wwfgtcshsn lngqyfrsip qqrqklkkgi fwktwrgryy plqattmliq |
| 361 | pmaaeaas |
| SMAD5 (Mothers against decapentaplegic homolog 5 also known as SMAD5; | |
| NP_001001419; NP_001001420; NP_005894) | |
| (SEQ ID NO: 25) | |
| 1 | mtsmaslfsf tspavkrllg wkqgdeeekw aekavdalvk klkkkkgame elekalsspg |
| 61 | qpskcvtipr sldgrlqvsh rkglphviyc rvwrwpdlqs hhelkpldic efpfgskqke |
| 121 | vcinpyhykr vespvlppvl vprhnefnpq hsllvqfrnl shnephmpqn atfpdsfhqp |
| 181 | nntpfplspn spyppspass typnspassg pgspfqlpad tpppaymppd dqmgqdnsqp |
| 241 | mdtsnnmipq impsissrdv qpvayeepkh wcsivyyeln nrvgeafhas stsvlvdgft |
| 301 | dpsnnksrfc lgllsnvnrn stientrrhi gkgvhlyyvg gevyaeclsd ssifvqsrnc |
| 361 | nfhhgfhptt vckipsscsl kifnnqefaq llaqsvnhgf eavyeltkmc tirmsfvkgw |
| 421 | gaeyhrqdvt stpcwieihl hgplqwldkv ltqmgsplnp issvs |
| TNFRSF12A (Tumor necrosis factor receptor superfamily member 12A also | |
| known as the TWEAK receptor (TWEAKR); NP_057723) | |
| (SEQ ID NO: 26) | |
| 1 | margslrrll rllvlglwla llrsvageqa pgtapcsrgs swsadldkcm dcascrarph |
| 61 | sdfclgcaaa ppapfrllwp ilggalsltf vlgllsgflv wrrcrrrekf ttpieetgge |
| 121 | gcpavaliq |
| SMOC2 (SPARC-related modular calcium-binding protein 2; NP_001159884; | |
| NP_071421) | |
| (SEQ ID NO: 27) | |
| 1 | mllpqlcwlp llagllppvp aqkfsaltfl rvdqdkdkdc sldcagspqk plcasdgrtf |
| 61 | lsrcefqrak ckdpqleiay rgnckdvsrc vaerkytqeq arkefqqvfi pecnddgtys |
| 121 | qvqchsytgy cwcvtpngrp isgtavahkt prcpgsvnek lpqregtgkt ddaaapalet |
| 181 | qpqgdeedia sryptlwteq vksrqnktnk nsvsscdqeh qsaleeakqp kndnvvipec |
| 241 | ahgglykpvq chpstgycwc vlvdtgrpip gtstryeqpk cdntarahpa kardlykgrq |
| 301 | lqgcpgakkh efltsvldal stdmvhaasd pssssgrlse pdpshtleer vvhwyfklld |
| 361 | knssgdigkk eikpfkrflr kkskpkkcvk kfveycdvnn dksisvqelm gclgvakedg |
| 421 | kadtkkrhtp rghaestsnr qprkqg |
| TNFRSF1A (Tumor necrosis factor receptor 1 (TNFR1), also known as tumor | |
| necrosis factor receptor superfamily member 1A (TNFRSF1A) and CD120a; | |
| NP_001056; NP_001333020; NP_001333021) | |
| (SEQ ID NO: 28) | |
| 1 | mglstvpdll lplvllellv giypsgvigl vphlgdrekr dsvcpqgkyi hpqnnsicct |
| 61 | kchkgtylyn dcpgpgqdtd crecesgsft asenhlrhcl scskcrkemg qveissctvd |
| 121 | rdtvcgcrkn qyrhywsenl fqcfncslcl ngtvhlscqe kqntvctcha gfflrenecv |
| 181 | scsnckksle ctklclpqie nvkgtedsgt tvllplviff glcllsllfi glmyryqrwk |
| 241 | sklysivcgk stpekegele gtttkplapn psfsptpgft ptlgfspvps stftssstyt |
| 301 | pgdcpnfaap rrevappyqg adpilatala sdpipnplqk wedsahkpqs ldtddpatly |
| 361 | avvenvpplr wkefvrrlgl sdheidrlel qngrclreaq ysmlatwrrr tprreatlel |
| 421 | lgrvlrdmdl lgcledieea lcgpaalppa psllr |
| ST6GAL1 (Beta-galactoside alpha-2,6-sialyltransferase 1; NP_003023; | |
| NP_775323; NP_775324; NP_001340845) | |
| (SEQ ID NO: 29) | |
| 1 | mihtnlkkkf sccvlvfllf avicvwkekk kgsyydsfkl qtkefqvlks lgklamgsds |
| 61 | qsvsssstqd phrgrqtlgs lrglakakpe asfqvwnkds ssknliprlq kiwknylsmn |
| 121 | kykvsykgpg pgikfsaeal rchlrdhvnv smvevtdfpf ntsewegylp kesirtkagp |
| 181 | wgrcavvssa gslkssqlgr eiddhdavlr fngaptanfq qdvgtkttir lmnsqlvtte |
| 241 | krflkdslyn egilivwdps vyhsdipkwy qnpdynffnn yktyrklhpn qpfyilkpqm |
| 301 | pwelwdilqe ispeeiqpnp pssgmlgiii mmtlcdqvdi yeflpskrkt dvcyyyqkff |
| 361 | dsactmgayh pllyeknlvk hlnqgtdedi yllgkatlpg frtihc |
| LAIR1 (Leukocyte-associated immunoglobulin-like receptor 1; NP_001275952; | |
| NP_001275954; NP_001275955; NP_001275956; NP_002278) | |
| (SEQ ID NO: 30) | |
| 1 | msphptallg lvlclaqtih tqedlprpsi saepgtvipl gshvtfvcrg pvgvqtfrle |
| 61 | rdsrstyndt edvsqaspse searfridsv regnaglyrc iyykppkwse qsdylellvk |
| 121 | gptqrpsdns hnehapasqg lkaehlyili gvsvvflfcl lllvlfclhr qnqikqgppr |
| 181 | skdeeqkpqq rpdlavdvle rtadkatvng lpekdretdt salaagssqe vtyaqldhwa |
| 241 | ltqrtaravs pqstkpmaes ityaavarh |
| CD74 (HLA class II histocompatibility antigen gamma chain also known as | |
| HLA-DR antigens-associated invariant chain or CD74; NP_001020329; | |
| NP_001020330; NP_004346; NP_001351012; NP_001351013) | |
| (SEQ ID NO: 31) | |
| 1 | mhrrrsrscr edqkpvmddq rdlisnneql pmlgrrpgap eskcsrgaly tgfsilvtll |
| 61 | lagqattayf lyqqqgrldk ltvtsqnlql enlrmklpkp pkpvskmrma tpllmqalpm |
| 121 | galpqgpmqn atkygnmted hvmhllqshw nwrtrllgwv |
| DPP7 (Dipeptidyl-peptidase 2; NP_037511) | |
| (SEQ ID NO: 32) | |
| 1 | mgsapwapvl llalglrglq agarrapdpg fqerffqqrl dhfnferfgn ktfpqrflvs |
| 61 | drfwvrgegp iffytgnegd vwafannsaf vaelaaerga llvfaehryy gkslpfgaqs |
| 121 | tqrghtellt veqaladfae llralrrdlg aqdapaiafg gsyggmlsay lrmkyphlva |
| 181 | galaasapvl avaglgdsnq ffrdvtadfe gqspkctqgv reafrqikdl flqgaydtvr |
| 241 | wefgtcqpls dekdltqlfm farnaftvla mmdypyptdf lgplpanpvk vgcdrllsea |
| 301 | qritglrala glvynasgse hcydiyrlyh scadptgcgt gpdarawdyq acteinltfa |
| 361 | snnvtdmfpd lpftdelrqr ycldtwgvwp rpdwlltsfw ggdlraasni ifsngnldpw |
| 421 | agggirrnls asviavtiqg gahhldlras hpedpasvve arkleatiig ewvkaarreq |
| 481 | qpalrggprl sl |
| MAPT (microtubule-associated protein tau; NP_001116538; NP_001116539; | |
| NP_001190180; NP_001190181; NP_005901) | |
| (SEQ ID NO: 33) | |
| 1 | maeprqefev medhagtygl gdrkdqggyt mhqdqegdtd aglkesplqt ptedgseepg |
| 61 | setsdakstp taedvtaplv degapgkqaa aqphteipeg ttaeeagigd tpsledeaag |
| 121 | hvtqepesgk vvqegflrep gppglshqlm sgmpgapllp egpreatrqp sgtgpedteg |
| 181 | grhapellkh qllgdlhqeg pplkgaggke rpgskeevde drdvdesspq dsppskaspa |
| 241 | qdgrppqtaa reatsipgfp aegaiplpvd flskvsteip asepdgpsvg rakgqdaple |
| 301 | ftfhveitpn vqkeqahsee hlgraafpga pgegpeargp slgedtkead lpepsekqpa |
| 361 | aaprgkpvsr vpqlkarmvs kskdgtgsdd kkaktstrss aktlknrpcl spkhptpgss |
| 421 | dpliqpsspa vcpeppsspk yvssvtsrtg ssgakemklk gadgktkiat prgaappgqk |
| 481 | gqanatripa ktppapktpp ssatkqvqrr pppagprser geppksgdrs gysspgspgt |
| 541 | pgsrsrtpsl ptpptrepkk vavvrtppks pssaksrlqt apvpmpdlkn vkskigsten |
| 601 | lkhqpgggkv qiinkkldls nvqskcgskd nikhvpgggs vqivykpvdl skvtskcgsl |
| 661 | gnihhkpggg qvevksekld fkdrvqskig sldnithvpg ggnkkiethk ltfrenakak |
| 721 | tdhgaeivyk spvvsgdtsp rhlsnvsstg sidmvdspql atladevsas lakqgl |
| TDGF1 (Teratocarcinoma-derived growth factor 1; NP_001167607; NP_003203) | |
| (SEQ ID NO: 34) | |
| 1 | maiskvfelg lvaglghqef arpsrgylaf rddsiwpqee pairprssqr vppmgiqhsk |
| 61 | elnrtcclng gtcmlgsfca cppsfygrnc ehdvrkencg svphdtwlpk kcslckcwhg |
| 121 | qlrcfpqafl pgcdglvmde hlvasrtpel ppsartttfm lvgiclsiqs yy |
| PMVK (Phosphomevalonate kinase; NP_001309940; NP_001309941; NP_006547; | |
| NP_001335625) | |
| (SEQ ID NO: 35) | |
| 1 | mpplhktvvy tgepqrsrlg advcavlrls gplkeqyaqe hglnfqrlld tstykeafrk |
| 61 | dmirwgeekr qadpgffcrk ivegisqpiw lvsdtrrvsd iqwfreayga vtqtvrvval |
| 121 | eqsrqqrgwv ftpgvddaes ecgldnfgdf dwvienhgve qrleeqlenl iefirsrl |
| CNTN2 (Contactin-2; NP_001333012; NP_005067) | |
| (SEQ ID NO: 36) | |
| 1 | mgtatrrkph lllvaavalv sssawssalg sqttfgpvfe dqplsvlfpe esteeqvlla |
| 61 | crarasppat yrwkmngtem klepgsrhql vggnlvimnp tkaqdagvyq clasnpvgtv |
| 121 | vsreailrfg flqefskeer dpvkahegwg vmlpcnppah ypglsyrwll nefpnfiptd |
| 181 | grhfvsqttg nlyiartnas dlgnysclat shmdfstksv fskfaqlnla aedtrlfaps |
| 241 | ikarfpaety alvgqqvtle cfafgnpvpr ikwrkvdgsl spqwttaept lqipsvsfed |
| 301 | egtyeceaen skgrdtvqgr iivqaqpewl kvisdteadi gsnlrwgcaa agkprptvrw |
| 361 | lrngeplasq nrvevlagdl rfsklsleds gmyqcvaenk hgtiyasael avqalapdfr |
| 421 | lnpvrrlipa arggeilipc qpraapkavv lwskgteilv nssrvtvtpd gtliirnisr |
| 481 | sdegkytcfa enfmgkanst gilsvrdatk itlapssadi nlgdnltlqc hashdptmdl |
| 541 | tftwtlddfp idfdkpgghy rrtnvketig dltilnaqlr hggkytcmaq tvvdsaskea |
| 601 | tvlvrgppgp pggvvvrdig dttiqlswsr gfdnhspiak ytlqartppa gkwkqvrtnp |
| 661 | aniegnaeta qvlgltpwmd yefrviasni lgtgepsgps skirtreaap svapsglsgg |
| 721 | ggapgelivn wtpmsreyqn gdgfgyllsf rrqgsthwqt arvpgadaqy fvysnesvrp |
| 781 | ytpfevkirs ynrrgdgpes ltalvysaee eprvaptkvw akgvsssemn vtwepvqqdm |
| 841 | ngillgyeir ywkagdkeaa adrvrtagld tsarvsglhp ntkyhvtvra ynragtgpas |
| 901 | psanattmkp pprrppgnis wtfsssslsi kwdpvvpfrn esavtgykml yqndlhltpt |
| 961 | lhltgknwie ipvpedigha lvqirttgpg gdgipaevhi vrnggtsmmv enmavrpaph |
| 1021 | pgtvishsva mliligslel |
| AMN (Amnionless; NP_112205) | |
| (SEQ ID NO: 37) | |
| 1 | mgvlgrvllw lqlcaltqav sklwvpntdf dvaanwsqnr tpcaggavef padkmvsvlv |
| 61 | qeghavsdml lpldgelvla sgagfgvsdv gshldcgage pavfrdsdrf swhdphlwrs |
| 121 | gdeapglffv daervpcrhd dvffppsasf rvglgpgasp vrvrsisalg rtftrdedla |
| 181 | vflasragrl rfhgpgalsv gpedcadpsg cvcgnaeaqp wicaallqpl ggrcpqaach |
| 241 | salrpqgqcc dlcgavvllt hgpafdlery rarildtflg lpqyhglqva vskvprssrl |
| 301 | readteiqvv lvengpetgg agrlaralla dvaengealg vleatmresg ahvwgssaag |
| 361 | laggvaaavl lallvllvap pllrragrlr wrrheaaapa gaplgfrnpv fdvtaseelp |
| 421 | lprrlslvpk aaadstshsy fvnplfagae aea |
| SLC39A5 (The serotonin transporter (SERT or 5-HTT) also known as the | |
| sodium-dependent serotonin transporter and solute carrier family 6 member | |
| 4 is a protein that in humans is encoded by the SLC6A4 gene; NP_001036) | |
| (SEQ ID NO: 38) | |
| 1 | mettplnsqk qlsacedged cqengvlqkv vptpgdkves gqisngysav pspgagddtr |
| 61 | hsipattttl vaelhqgere twgkkvdfll svigyavdlg nvwrfpyicy qngggafllp |
| 121 | ytimaifggi plfymelalg qyhrngcisi wrkicpifkg igyaiciiaf yiasyyntim |
| 181 | awalyyliss ftdqlpwtsc knswntgnct nyfsednitw tlhstspaee fytrhvlqih |
| 241 | rskglqdlgg iswqlalcim liftviyfsi wkgvktsgkv vwvtatfpyi ilsvllvrga |
| 301 | tlpgawrgvl fylkpnwqkl letgvwidaa aqiffslgpg fgvllafasy nkfnnncyqd |
| 361 | alvtsvvncm tsfvsgfvif tvlgymaemr nedvsevakd agpsllfity aeaianmpas |
| 421 | tffaiifflm litlgldstf aglegvitav ldefphvwak rrerfvlavv itcffgslvt |
| 481 | ltfggayvvk lleeyatgpa vltvalieav avswfygitq fcrdvkemlg fspgwfwric |
| 541 | wvaisplfll fiicsflmsp pqlrlfqyny pywsiilgyc igtssficip tyiayrliit |
| 601 | pgtfkeriik sitpetptei pcgdirlnav |
| DGKZ (Diacylglycerol kinase zeta; NP_001099010; NP_001186195; NP_001186196; | |
| NP_001186197; NP_003637) | |
| (SEQ ID NO: 39) | |
| 1 | metffrrhfr gkvpgpgegq qrpssvglpt gkarrrspag qassslaqrr rssaqlqgcl |
| 61 | lscgvraqgs srrrsstvpp scnprfivdk vltpqpttvg aqllgaplll tglvgmneee |
| 121 | gvqedvvaea ssaiqpgtkt pgpppprgaq pllplprylr rasshllpad avydhalwgl |
| 181 | hgyyrrlsqr rpsgqhpgpg grrasgttag tmlptrvrpl srrrqvalrr kaagpqawsa |
| 241 | llakaitksg lqhlappppt pgapcseser qirstvdwse satygehiwf etnvsgdfcy |
| 301 | vgeqycvarm lksvsrrkca ackivvhtpc ieqlekinfr ckpsfresgs rnvreptfvr |
| 361 | hhwvhrrrqd gkcrhcgkgf qqkftfhske ivaiscswck qayhskvscf mlqqieepcs |
| 421 | lgvhaavvip ptwilrarrp qntlkaskkk krasfkrkss kkgpeegrwr pfiirptpsp |
| 481 | lmkpllvfvn pksggnqgak iiqsflwyln prqvfdlsqg gpkealemyr kvhnlrilac |
| 541 | ggdgtvgwil stldqlrlkp pppvailplg tgndlartln wgggytdepv skilshveeg |
| 601 | nvvqldrwdl haepnpeagp edrdegatdr lpldvfnnyf slgfdahvtl efhesreanp |
| 661 | ekfnsrfrnk mfyagtafsd flmgsskdla khirvvcdgm dltpkiqdlk pqcvvflnip |
| 721 | rycagtmpwg hpgehhdfep qrhddgylev igftmtslaa lqvgghgerl tqcrevvltt |
| 781 | skaipvqvdg epcklaasri rialrnqatm vqkakrrsaa plhsdqqpvp eqlriqvsrv |
| 841 | smhdyealhy dkeqlkeasv plgtvvvpgd sdlelcrahi erlqqepdga gaksptcqkl |
| 901 | spkwcfldat tasrfyridr aqehlnyvte iaqdeiyild pellgasarp dlptptsplp |
| 961 | tspcsptprs lqgdaappqg eelieaakrn dfcklqelhr aggdlmhrde qsrtllhhav |
| 1021 | stgskdvvry lldhappeil daveengetc lhqaaalgqr tichyiveag aslmktdqqg |
| 1081 | dtprqraeka qdtelaayle nrqhyqmiqr edqetav |
| AXIN1 (Axin-1; NP_003493; NP_851393) | |
| (SEQ ID NO: 40) | |
| 1 | mniqeqgfpl dlgasfteda prppvpgeeg elvstdprpa sysfcsgkgv gikgetstat |
| 61 | prrsdldlgy epegsasptp pylkwaeslh sllddqdgis lfrtflkqeg cadlldfwfa |
| 121 | ctgfrklepc dsneekrlkl araiyrkyil dnngivsrqt kpatksfikg cimkqlidpa |
| 181 | mfdqaqteiq atmeentyps flksdiyley trtgsespkv csdqssgsgt gkgisgylpt |
| 241 | lnedeewkcd qdmdeddgrd aappgrlpqk llletaaprv sssrrysegr efrygswrep |
| 301 | vnpyyvnagy alapatsand seqqslssda dtlsltdssv dgippyrirk qhrremqesv |
| 361 | qvngrvplph iprtyrvpke vrvepqkfae elihrleavq rtreaeekle erlkrvrmee |
| 421 | egedgdpssg ppgpchklpp apawhhfppr cvdmgcaglr daheenpesi ldehvqrvlr |
| 481 | tpgrqspgpg hrspdsghva kmpvalggaa sghgkhvpks gakldaaglh hhrhvhhhvh |
| 541 | hstarpkeqv eaeatrraqs sfawglephs hgarsrgyse svgaapnasd glahsgkvgv |
| 601 | ackrnakkae sgksastevp gasedaeknq kimqwiiege keisrhrrtg hgssgtrkpq |
| 661 | phensrplsl ehpwagpqlr tsvqpshlfi qdptmpphpa pnpltqleea rrrleeeekr |
| 721 | asrapskqry vqevmrrgra cvrpacapvl hvvpavsdme lsetetrsqr kvgggsaqpc |
| 781 | dsivvayyfc gepipyrtlv rgravtlgqf kelltkkgsy ryyfkkvsde fdcgvvfeev |
| 841 | redeavlpvf eekiigkvek vd |
| CEBPB (CCAAT/enhancer-binding protein beta; NP_001272807; NP_001272808; | |
| NP_005185) | |
| (SEQ ID NO: 41) | |
| 1 | mevanfyyea dclaaayggk aapaappaar pgprppagel gsigdherai dfspyleplg |
| 61 | apqapapata tdtfeaappa papapassgq hhdflsdlfs ddyggknckk paeygyvslg |
| 121 | rlgaakgalh pgcfaplhpp pppppppael kaepgfepad ckrkeeagap gggagmaagf |
| 181 | pyalraylgy qavpsgssgs lstssssspp gtpspadaka pptacyagaa papsqvkska |
| 241 | kktvdkhsde ykirrernni avrksrdkak mrnletqhkv leltaenerl qkkveqlsre |
| 301 | lstlrnlfkq lpepllassg hc |
| ZBTB16 (Zinc finger and BTB domain-containing protein 16; NP_001018011; | |
| NP_005997; NP_001341679; NP_001341680; NP_001341681) | |
| (SEQ ID NO: 42) | |
| 1 | mdltkmgmiq lqnpshptgl lckanqmrla gtlcdvvimv dsqefhahrt vlactskmfe |
| 61 | ilfhrnsqhy tldflspktf qqileyayta tlqakaedld dllyaaeile ieyleeqclk |
| 121 | mletiqasdd ndteatmadg gaeeeedrka rylknifisk hsseesgyas vagqslpgpm |
| 181 | vdqspsvsts fglsamsptk aavdslmtig qsllqgtlqp pagpeeptla gggrhpgvae |
| 241 | vktemmqvde vpsqdspgaa essisggmgd kveergkegp gtptrssvit sarelhygre |
| 301 | esaeqvpppa eagqaptgrp ehpapppekh lgiysvlpnh kadavlsmps svtsglhvqp |
| 361 | alavsmdfst yggllpqgfi qrelfsklge lavgmksesr tigeqcsvcg velpdneave |
| 421 | qhrklhsgmk tygcelcgkr fldslrlrmh llahsagaka fvcdqcgaqf skedalethr |
| 481 | qthtgtdmav fcllcgkrfq aqsalqqhme vhagvrsyic secnrtfpsh talkrhlrsh |
| 541 | tgdhpyecef cgscfrdest lkshkrihtg ekpyecngcg kkfslkhqle thyrvhtgek |
| 601 | pfecklchqr srdysamikh lrthngaspy qcticteycp slssmqkhmk ghkpeeippd |
| 661 | wriektylyl cyv |
| CHI3L1 (Chitinase-3-like protein 1; NP_001267; NP_001267.2) | |
| (SEQ ID NO: 43) | |
| 1 | mgvkasqtgf vvlvllqccs ayklvcyyts wsqyregdgs cfpdaldrfl cthiiysfan |
| 61 | isndhidtwe wndvtlygml ntlknrnpnl ktllsvggwn fgsqrfskia sntqsrrtfi |
| 121 | ksvppflrth gfdgldlawl ypgrrdkqhf ttlikemkae fikeaqpgkk qlllsaalsa |
| 181 | gkvtidssyd iakisqhldf isimtydfhg awrgttghhs plfrgqedas pdrfsntdya |
| 241 | vgymlrlgap asklvmgipt fgrsftlass etgvgapisg pgipgrftke agtlayyeic |
| 301 | dflrgatvhr ilgqqvpyat kgnqwvgydd qesvkskvqy lkdrqlagam vwaldlddfq |
| 361 | gsfcgqdlrf pltnaikdal aat |
| DUOX2 (Dual oxidase 2; NP_054799; NP_001350640) | |
| (SEQ ID NO: 44) | |
| 1 | mlrarpealm llgalltgsl gpsgnqdals lpwevqrydg wfnnlrhher gavgcrlqrr |
| 61 | vpanyadgvy qaleepqlpn prrlsnaatr giaglpslhn rtvlgvffgy hvlsdvvsve |
| 121 | tpgcpaefln irippgdpvf dpdqrgdvvl pfqrsrwdpe tgrspsnprd lanqvtgwld |
| 181 | gsaiygsshs wsdalrsfsg gqlasgpdpa fprdsqnpll mwaapdpatg qngprglyaf |
| 241 | gaergnrepf lqalgllwfr yhnlwaqrla rqhpdwedee lfqharkrvi atyqniavye |
| 301 | wlpsflqktl peytgyrpfl dpsispefvv aseqffstmv ppgvymrnas chfrkvlnkg |
| 361 | fqssqalrvc nnywirenpn lnstqevnel llgmasqise lednivvedl rdywpgpgkf |
| 421 | srtdyvassi qrgrdmglps ysqallafgl diprnwsdln pnvdpqvlea taalynqdls |
| 481 | qlelllggll eshgdpgplf saivldqfvr lrdgdrywfe ntrnglfskk eiedirnttl |
| 541 | rdvlvavini dpsalqpnvf vwhkgapcpq pkqlttdglp qcapltvldf fegsspgfai |
| 601 | tiialcclpl vslllsgvva yfrgrehkkl qkklkesvkk eaakdgvpam ewpgpkerss |
| 661 | piiiqllsdr clqvlnrhlt vlrvvqlqpl qqvnlilsnn rgcrtlllki pkeydlvllf |
| 721 | sseeergafv qqlwdfcvrw alglhvaems ekelfrkavt kqqrerilei ffrhlfaqvl |
| 781 | dinqadagtl pldssqkvre altcelsrae faeslglkpq dmfvesmfsl adkdgngyls |
| 841 | frefldilvv fmkgspedks rlmftmydld engflskdef ftmmrsfiei snnclskaql |
| 901 | aevvesmfre sgfqdkeelt wedfhfmlrd hdselrftql cvkggggggn girdifkqni |
| 961 | scrvsfitrt pgershpqgl gppapeapel ggpglkkrfg kkaavptprl ytealqekmq |
| 1021 | rgflaqklqq ykrfvenyrr hivcvaifsa icvgvfadra yyygfaspps diaqttlvgi |
| 1081 | ilsrgtaasv sfmfsyillt mcrnlitflr etflnryvpf daavdfhrwi amaavvlail |
| 1141 | hsaghavnvy ifsvsplsll acifpnvfvn dgsklpqkfy wwffqtvpgm tgvllllvla |
| 1201 | imyvfashhf rrrsfrgfwl thhlyillya lliihgsyal iqlptfhiyf lvpaiiyggd |
| 1261 | klvslsrkkv eisvvkaell psgvtylqfq rpqgfeyksg qwvriaclal gtteyhpftl |
| 1321 | tsaphedtls lhiravgpwt trlreiyssp kgngcagypk lyldgpfgeg hqewhkfevs |
| 1381 | vlvgggigvt pfasilkdlv fksslgsqml ckkiyfiwvt rtqrqfewla diiqeveend |
| 1441 | hqdlvsvhiy vtqlaekfdl rttmlyicer hfqkvlnrsl ftglrsithf grppfepffn |
| 1501 | slqevhpqvr kigvfscgpp gmtknvekac qlvnrqdrah fmhhyenf |
| CST3 (Cystatin C or cystatin 3; NP_000090; NP_001275543) | |
| (SEQ ID NO: 45) | |
| 1 | magplrapll llailavala vspaagsspg kpprlvggpm dasveeegvr raldfavgey |
| 61 | nkasndmyhs ralqvvrark qivagvnyfl dvelgrttct ktqpnldncp fhdqphlkrk |
| 121 | afcsfqiyav pwqgtmtlsk stcqda |

Without further elaboration, it is believed that one skilled in the art, using the preceding description, can utilize the present invention to the fullest extent. The following examples are illustrative only, and not limiting of the remainder of the disclosure in any way whatsoever.

### Example

The following examples are put forth so as to provide those of ordinary skill in the art with a complete disclosure and description of how the compounds, compositions, articles, devices, and/or methods described and claimed herein are made and evaluated and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Introduction

Gene expression profiling (GEP) has been shown to yield superior prognostic accuracy in predicting metastasis in UM compared to clinical, histopathologic, and chromosomal features (3-6), as well as compared to the widely used TNM staging (7). This method has been validated prospectively and is now being used by clinicians to stratify the risk of metastasis by assigning UM patients to two highly prognostic molecular classes: class 1 (low metastatic risk) and class 2 (high metastatic risk) (3,8). An optimized test has been developed using a 15-gene expression profile assay that is performed on a microfluidics polymerase chain reaction platform, allowing it to detect small quantities of tumor RNA from both fine needle aspiration samples and surgically resected specimens (3). This test's accuracy and high technical reliability has been well established in several retrospective and prospective studies (3,4,9-14), and this test is clinically used by physicians to guide metastatic surveillance recommendations (12,15,16).

While tumor biopsies can provide reliable assessment risk of metastatic disease through molecular testing, they are subject to risks of retinal detachment and tumor dissemination (17). In addition, results from a recent study that consecutively sampled two sites of the same tumor via fine needle aspiration biopsy (FNAB) show that discordance between GEP results may exist in 11-16% of cases (18). Their data suggests that a single-site FNAB with a low metastasis result may be misleading in a small minority of patients, as the site of the tumor sampled may be populated by lower-metastatic potential cells than other cell populations in a heterogenous tumor. For these reasons, there is a benefit to establish an ocular liquid biopsy platform for proteomic analysis of UM tumors that can be both less invasive and more representative of the heterogenous subpopulations of tumor cells.

In addition to the limitations of tumor biopsies, genetic analysis does not predict protein expression and posttranslational modification, resulting in an incomplete understanding of tumor phenotype (19). Therefore, the analysis of the proteome of AH from UM patients has the potential to improve diagnosis, prognosis, surveillance, and treatment of patients, through providing a more precise characterization of the tumor phenotype. Both the vitreous and aqueous humor have been used as ocular liquid biopsy platforms to study proteomics in UM patients. A recent study of Velez et al. has identified proteomic biomarkers in the vitreous humor of UM patients that were associated with metastatic risk, with their results suggesting mechanisms of tumor proliferation and approaches for adjuvant therapy and metastatic risk surveillance (17). Another study by Wierenga et al. identified distinct differences between cytokines in different aqueous humor samples of UM patients, which were then allocated into three different prognostic tumor clusters (20). A recent review by Heiferman et al. describes in further detail the recent advances in proteomics in AH, VH, and tumor of UM patients (20).

Despite the recent advancements in liquid biopsy proteomics in UM patients, there is still not a clinical assay in use that can identify biomarkers at the time of diagnosis, during, and after treatment. Provided herein is the first to analyze AH from treatment naïve eyes to identify therapeutic prognostic biomarkers in UM patients, by comparing diagnostic AH samples collected from GEP1 and GEP2 UM eyes. The potential of the AH as an organ specific liquid biopsy for UM through the presence of tumor-derived cell-free DNA was previously shown (21). In this study, the AH platform is further developed through analysis of how classification of differentially expressed protein biomarkers can compare to the widely used GEP classifications. The AH protein expression levels with metastatic potential at the diagnostic stage were correlated.

### Materials and Methods

This investigation was a case series study at a tertiary care hospital (University of Southern California Roski Eye Institute). Samples were taken between August 2020 and May 2021.

### Patient Clinical Characteristics and Demographics

This study included a convenience sample of 20 UM patients at the University of Southern California Roski Eye Institute from whom written informed consent for an AH sample was obtained. All samples consisted of ~0.1 mL of AH extracted via clear cornea paracentesis at the end of surgery for brachytherapy plaque placement. 20 AH samples from 20 UM eyes were included. Proteomics results were coded and maintained separately from clinical data and thus did not alter patient treatment for all participants.

### Specimen Collection and Storage

A clear corneal paracentesis with a 30-gauge needle was performed to extract ~0.1 mL of AH from UM eyes during clinically indicated surgery to treat UM. The extraction method has been described in detail and published previously by for specimen collection from retinoblastoma eyes (27). Briefly, needles only entered the anterior chamber via the clear cornea at the limbus and did not make contact with the iris, lens, vitreous, or UM tumor. Samples were stored on dry ice immediately and transferred to −80° C. within hours of extraction. Routine FNAB with either a 25- or 27-gauge needle was conducted on 16 patients for mutational analysis and 17 patients for GEP and PRAME status which was performed at Castle Biosciences (Phoenix, AZ, USA).

### Quantification of Protein Expression by Proximity Extension Assay (PEA)

AH samples were analyzed for 1472 proteins using the Olink Explore PEA assay. Briefly, protein-specific antibodies tagged with DNA sequences were used, followed by a NGS PEA gives abundance levels for each protein measured as NPX values (Normalized Protein expression) on a log 2 scale. The PEA assay was chosen over other protein assays due to several factors. Other approaches for protein detection, such as mass spectroscopy and ELISA, have some disadvantages when measuring proteins of low abundance: ELISA is not scalable to measure>90 proteins at a time per sample, and mass spectroscopy favors highly abundant proteins. With PEA, it is possible to measure proteins of low abundance with high sensitivity and specificity while enabling high throughput using a minimal amount of sample. This makes the assay ideal for measuring a high range of proteins in a large number of samples.

### Gene Ontology Analysis and Annotations

QIAGEN's Ingenuity® Pathway Analysis (IPA®, QIAGEN, www.qiagen.com/ingenuity) software was used for functional annotation pathway analysis and upstream regulatory analysis (URA) of the 45 DEPs (PMID: 26202100). Activation z-scores and the corresponding p values were generated by IPAR. Z-score predicts the activation (positive value) or inhibition (negative value) of a canonical pathway/upstream regulator. Absolute z-score values≥2 are considered significant.

### Statistical Analysis

Continuous variables were represented as the mean±standard deviation (S.D.), non-normally distributed variables were compared by the Mann-Whitney U test. Categorical variables were compared using the Fisher's exact test or linear-by-linear association test as indicated in Table 1. All statistical tests were two-tailed, and p<0.05 was considered statistically significant. p-values are represented as: *, p<0.05; **, p<0.01; ***, p<0.001; ns, non-significant. All statistical analyses and plots were conducted using the Prism 8 (GraphPad, San Diego, CA, USA).

### Results

### Patient Clinical Characteristics and Demographics

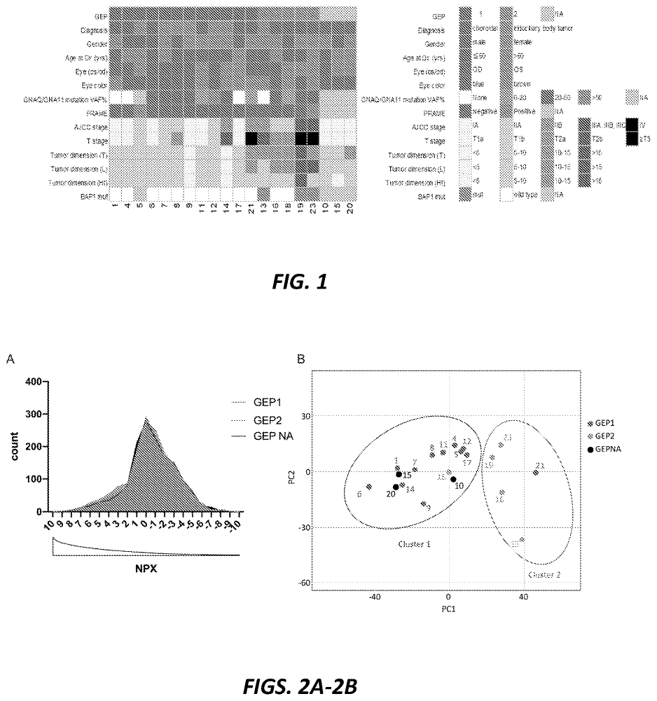

Twenty UM AH samples collected at diagnosis, prior to any therapy, from 20 UM patients were analyzed. Patient demographics and clinical characteristics are summarized in FIG. 1. BAP1 mutation, preferentially expressed antigen in melanoma (PRAME) status, and GEP class is defined from clinical indicated tumor biopsy when available. A total of twelve (60%) GEP1, five (25%) GEP2 and three (15%) patients without available tumor biopsy were included. All GEP1 tumors were AJCC stage I or II, while 2/5 (40%) of GEP2 tumors were more advanced (p=0.012; Table 1). A significant number of GEP2 tumors were diagnosed at a more advanced clinical tumor stage than GEP 1 tumors (p=0.007; Table 1). None of the GEP1 tumors harbor a BAP1 mutation but 3/5 (60%) of GEP2 tumors had mutant BAP1, a known poor prognostic marker (p=0.018; Table 1).

TABLE 1
Univariate comparison of clinical characteristics
between GEP1 and GEP2 UM patients

| | GEP1, | GEP2, | |
|---|---|---|---|
| Characteristic | n = 12 | n = 5 | p value |
| Sex (Fisher), n (%) | | | 0.600 |
| Females | 7 (58.3) | 4 (80) | |
| Males | 5 (41.7) | 1 (20) | |
| Eye (Fisher), n (%) | | | 0.620 |
| OD | 7 (58.3) | 2 (40.0) | |
| OS | 5 (41.7) | 3 (60.0) | |
| Age at diagnosis, mean | 53.3 (15.5) | 62.8 (12.7) | 0.183 |
| (±SD) (MWU) | | | |
| Eye Color (Fisher), n (%) | | | 0.620 |
| Light (blue, gray, green, | 7 (58.3) | 2 (40.0) | |
| hazel) | | | |
| Dark (brown) | 5 (41.7) | 3 (60.0) | |
| Anterior Involvement (Iris | | | 0.338 |
| and/or ciliary body) (Fisher), | | | |
| n (%) | | | |
| Yes | 6 (50.0) | 1 (20.0) | |
| No | 6 (50.0) | 4 (80.0) | |
| AJCC Stage (Linear-by- | | | 0.012 |
| Linear association), n (%) | | | |
| I | 6 (50.0) | 0 (0) | |
| IIA | 4 (33.3) | 2 (40.0) | |
| IIB | 2 (16.7) | 1 (20.0) | |
| IIIA, IIIB, IIIC | 0 (0) | 2 (40.0) | |
| IV | 0 (0) | 0 (0) | |
| PRAME Status, known in 17 | | | 0.515 |
| cases (Fisher), n (%) | | | |
| Negative | 11 (91.7) | 4 (80.0) | |
| Positive | 1 (8.3) | 1 (20.0) | |
| GEP Class, known in 17 | | | <0.001 |
| cases (Fisher), n (%) | | | |
| 1 | 12 (100.0) | 0 (0) | |
| 2 | 0 (0) | 5 (80.0) | |
| Tumor Stage (Linear-by- | | | 0.007 |
| Linear association), n (%) | | | |
| T1 | 9 (75.0) | 0 (0) | |
| T2 | 2 (16.7) | 3 (60.0) | |
| T3 | 1 (8.3) | 1 (20.0) | |
| T4 | 0 (0) | 1 (20.0) | |
| BAP1 mutation status, know | | | 0.018 |
| in 16 cases (Fisher), n (%) | | | |
| Mutation | 0 (0) | 3 (60.0) | |
| Wild type | 11 (100.0) | 2 (40.0) | |

AJCC, American Joint Committee in Cancer;
Fisher, Fisher's exact test;
GEP, gene expression profile;
MWU, Mann-Whitney U test;
PRAME, preferentially expressed antigen in melanoma;
SD, standard deviation

### Target Protein Data Distribution

Using a multiplex proximity extension assay (PEA) platform by Olink, expression levels of 1472 proteins were analyzed from each AH sample. After excluding three low-quality targets, expression levels of 1469 proteins were normalized for variation using internal and external controls and the generated normalized protein expression units (NPX) were further analyzed. By comparing NPX data structure, no significant difference of NPX distribution (FIG. 2) between GEP classes were observed, suggesting normalized protein expression units were evenly distributed. Principle components assay (PCA) of the 1469 target NPX values showed two distinct clusters, with 15 AH in cluster 1 and 5 AH in cluster 2 (FIG. 2B). Comparing with FNAB-derived GEP classifications, 11/12 GEP1 cases (91.67%) lands in PEA-cluster 1, and 4/5 GEP2 cases (80.00%) lands in PEA-cluster 2. All 3 GEP unknown cases are included in PEA-cluster 1 (FIG. 2B).

### Differentially Expression Proteins Between GEP2 and GEP1 AH Samples

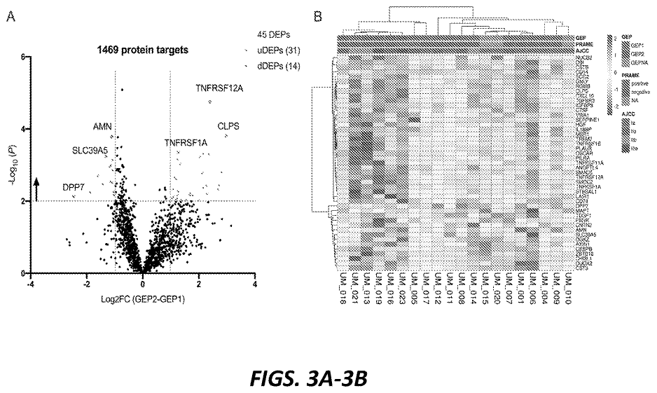

To identify the AH differentially expressed proteins (DEPs) related to GEP classes, NPX values of GEP2 samples were compared with GEP1 samples by setting the threshold that p value is smaller than 0.01 and log 2 (fold change) is greater than 1. Among the identified 45 DEPs, 31/45 (68.9%) and 14/45 (31.1%) were found to be upregulated and downregulated in GEP2, respectively (FIG. 3A). To analyze the discrimination power of the DEPs, an unsupervised clustering of all 20 AH samples was performed using the 45 DEPs. 5/5 GEP2 samples formed a distinct cluster on the left side of the heatmap, with only one GEP1 case (UM_021) clustered with GEP2 samples (FIG. 3B). 9/12 GEP1 and 3/3 GEP NA samples formed the right side of the heatmap, suggesting all 3 GEP NA cases (UM_010,015 and 020) may be GEP1 diseases (FIG. 3B). Two GEP1 cases (UM_005 and 017) clustered closer to left side, suggesting they might be in transitional stages from benign diseases to more advanced diseases.

### Biological Significance of the DEPs

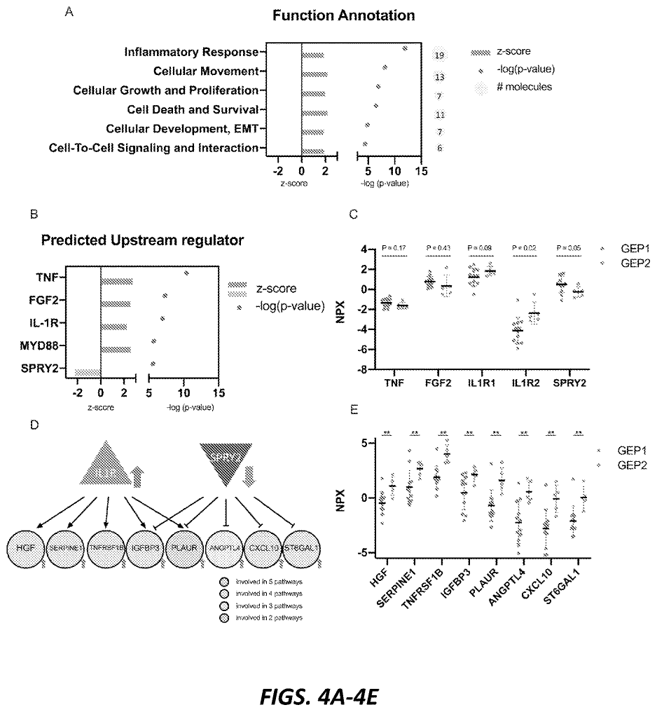

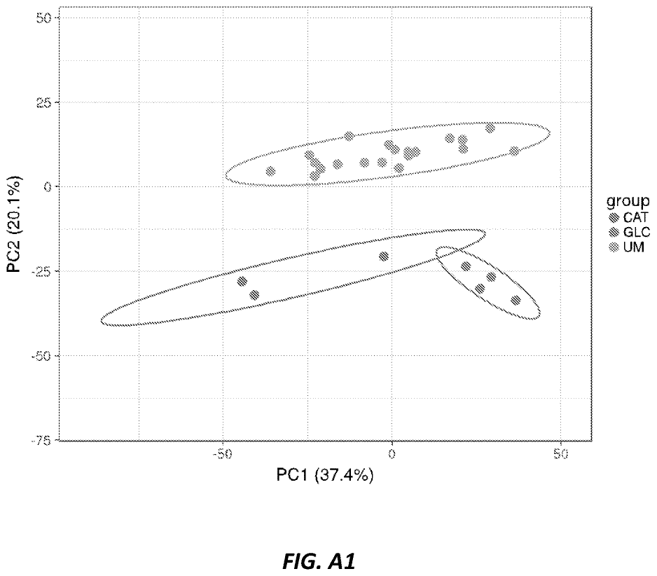

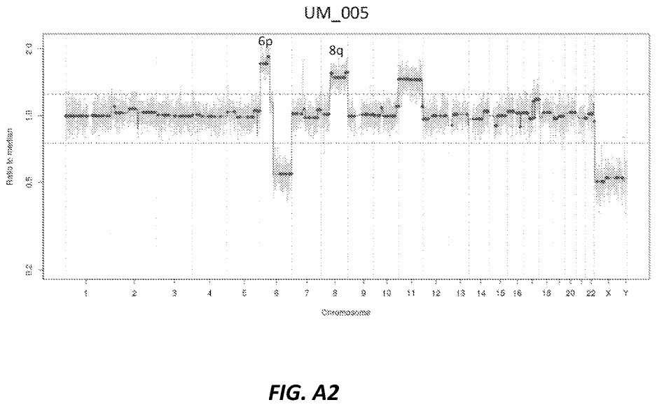

To determine the biological relevance of the 45 DEPs, pathway analysis was conducted to identify the important cellular function these DEPs may contribute. By performing Qiagen Ingenuity Pathway Analysis (IPA) on the 45 DEPs, inflammatory response, cellular growth-(cellular growth and proliferation and cell death and survival) and cell motility-(cellular movement and epithelial to mesenchymal transition (EMT)) related pathways were significantly identified (FIG. 4A). Aan upstream regulator analysis (URA) was performed to identify the potential upstream target that has been experimentally verified to affect the 45 DEP expression. The most significant upstream regulators of the 45 DEPs were TNF, FGF2, IL-1 receptor, MYD88 and SPRY2 (FIG. 4B, −log p value>5). In the PEA platform, upregulation of IL-1 receptor 2 (p=0.020, FIG. 4C) and downregulation of SPRY2 (p=0.052, FIG. 4C) were found to be concordant with the URA z-score (FIG. 4B) while MYD88 was not in our 1472 protein panel. The downstream targets of IL-1 receptor and SPRY2 within the 45 DEPs are listed in FIG. 4D. To note, co-targeting of IGFBP3 and PLAUR by both IL-1 receptor and SPRY2 (FIG. 4D) may lead to the subsequently activation of their protein expression in GEP2 samples (FIG. 4E).

### Discussion

While GEP profiling is a clinically validated and widely established method to stratify risk of metastasis in UM patients, it requires intraocular tumor biopsy. This is limited by small tumor size, tumor heterogeneity, as well as risk of retinal hemorrhage, bleeding, and tumor dissemination. Provided herein it is demonstrated that with minimally invasive aqueous humor extraction from UM eyes, a metastatic risk stratification similar to GEP classification by FNAB is provided. The first step to achieve this goal was to identify if there are differential patterns of molecules existing in diagnostic UM AH samples. Due to the low abundance and highly fragmented status of nucleic acid in pre-radiation AHs, the differential expression of proteins (DEPs) between GEP classes from AH of 20 treatment-naïve eyes was determined in this study.

The use of proximity extension assay (PEA)-derived multiplexed technology enabled the comparison of 1472 protein targets in small volume of AH samples. 45 differentially expressed proteins were identified when comparing quantities of these 1472 targets in the AH from 12 GEP1 patients and 5 GEP2 patients, which were then correlated with clinical features. The 3 samples for which the GEP was unknown, due to sample availability, are presumed to be likely GEP1 due to their 45 DEP-based classification. Notably, while these tumors were too small to biopsy, they were able to be classified into a presumed GEP class based on their protein expression from this assay.

Among these 17 cases with known GEP class, all GEP class 2 cases were identified by this DEP-clustering, while three GEP class 1 cases were not tightly clustered together with other cases. UM_021 (GEP1), which sits in the middle of other GEP2 cases, is stage T3 with 8.28 mm tumor size in height. It has been shown that incorporation of tumor size improves the prognostic ability of GEP classifications in patients with posterior UM so it is possible that there are clonal expression differences in this large tumor so that the AH analysis identifies this large tumor as class 2, while the tumor biopsy identified it as class 1. Genomic analysis can be done via the AH in addition to protein expression analysis to identify other markers of metastatic risk. Somatic copy number alteration profiling for UM_005 identified a gain in 8q (Figure A2), which is known to correlate with higher rates of metastasis and risk of mortality (22,23). SCNA analysis also revealed a gain of chromosome 6p. The combination of chromosome 8q and 6p gain places the patient in The Cancer Genome Atlas (TCGA) class B, again suggesting increased risk of metastasis (24). Further, the patient is PRAME positive, suggesting this patient has three risk factors for poor prognosis despite being GEP1 from tumor biopsy. There were no positive UM_SCNAs identified in UM_017 due to the low quantity of DNA available for genomic analyses. While these are a small number of cases, it suggests that AH DEP analysis, especially when combined with genomic analysis, may have increased utility in capturing molecular biomarkers indicating poor prognosis.

The cellular functions of these DEPs were identified to be associated with cell growth and motility. Upstream regulator analysis showed that the significant upstream regulators of the 45 DEPs were TNF, FGF2, IL-1 receptor, MYD88 and SPRY2. Both the upregulation of IL-1 and the downregulation of SPRY2 were also shown in the Olink PEA-derived platform. The upregulation of IL-1 has been shown in other cancers, including breast, colon, head and neck, lung, pancreas, and melanomas, with patients with high levels of IL-1 having generally poor prognosis (25). The downregulation of SPRY2 has been shown in chronic lymphocytic leukemia, non-small cell lung cancer, hepato-cellular carcinoma, breast cancer, and prostate cancers (26), highlighting its tumor sup-pressor function.

Prior studies of UM patients by Velez et al. and Wierenga et al. that analyzed the vitreous and aqueous humor respectively, have identified additional prognostic protein biomarkers. Velez's work identified protein signatures in the vitreous humor that correlated with GEP and PRAME. Although similar elevated trends of their targets (SCFR/c-KIT, HGFR/c-MET and SIGLEC6) were found in the AH of GEP2 eyes, no statistically significant difference was observed. It might be due to the fact that they compared AJCC III-IV vs I-II stage tumors at validation while only two AJCC III stage tumors were in the instant cohort. While their approach using the vitreous humor and the present using the aqueous humor both aim to provide prognostic biomarkers via a minimally invasive method for real-time intraocular assessment of UM, the instant method has the potential benefit of being even less invasive. In contrast, after analyzing 92 cytokines in AH from 84 enucleated UM eyes showing distinct patterns, Wierenga et al. allocated these samples into three different prognostic tumor clusters that were analyzing metastatic death, as opposed to intraocular metastatic risk. Still, high concordance was observed on over-lapped targets with the analyses provided herein, suggesting that one could converge efforts provided herein with their PEA-based proteomic dataset for future combined ocular and life mortality analysis.

Identification of biomarkers, such as SPRY2 and IL-1 from the upstream regulator analysis, suggests mechanisms of tumor proliferation and approaches for adjuvant therapy, due to the potential of such proteins to become eventual targets for drug re-positioning. As the use of different assays on different liquid biopsy origins (AH or VH) have provided distinct prognostic biomarkers, there is a potential for additional studies with similar aims to continue identifying more biomarkers, which may increase the likelihood of identifying a suitable target for pharmaceuticals for these patients. Moreover, in vivo proteomic profiling from AH can suggest biologically plausible mechanisms for tumor proliferation and suggest rational approaches for adjuvant therapy and metastatic risk surveillance.

With benefits of being eye-specific, minimally invasive, and conducive for treatment monitoring as well as longitudinal minimal residual disease detection and metastatic surveillance, aqueous humor liquid biopsies offer a desirable alternative to tissue biopsies. Especially in cases in which the tumor is too small to biopsy, analyzing protein expression in the aqueous humor has the potential to provide an alternative method of classifying such tumors into presumed GEP classes. This is clearly illustrated by the three cases included in the analysis which were too small to biopsy (posterior tumors with <2.5 mm height) yet yielded meaningful proteomic signals in AH analysis.

The results demonstrate that proteomic analysis of AH has a role for clinical utility as a surrogate to tumor biopsy-based staging, such as GEP classification, for determining metastatic potential.

### BIBLIOGRAPHY

1. Jager, M. J.; Shields, C. L.; Cebulla, C. M.; Abdel-Rahman, M. H.; Grossniklaus, H. E.; Stern, M.-H.; Carvajal, R. D.; Belfort, R. N.; Jia, R.; Shields, J. A.; et al. Uveal Melanoma. Nat. Rev. Dis. Primer 2020, 6, 24, doi: 10.1038/s41572-020-0158-0.
2. Kujala, E.; Mäkitie, T.; Kivelä, T. Very Long-Term Prognosis of Patients with Malignant Uveal Melanoma. Invest. Ophthalmol. Vis. Sci. 2003, 44, 4651-4659, doi: 10.1167/iovs.03-0538.
3. Onken, M. D.; Worley, L. A.; Char, D. H.; Augsburger, J. J.; Correa, Z. M.; Nudleman, E.; Aaberg, T. M.; Altaweel, M. M.; Bardenstein, D. S.; Finger, P. T.; et al. Collaborative Ocular Oncology Group Report No. 1: Prospective Validation of a Multi-Gene Prognostic Assay in Uveal Melanoma. Ophthalmology 2012, 119, 1596-1603, doi: 10.1016/j.ophtha.2012.02.017.
4. Corrêa, Z. M.; Augsburger, J. J. Independent Prognostic Significance of Gene Expression Profile Class and Largest Basal Diameter of Posterior Uveal Melanomas. Am. J. Ophthalmol. 2016, 162, 20-27.el, doi: 10.1016/j.ajo.2015.11.019.
5. Petrausch, U.; Martus, P.; Tönnies, H.; Bechrakis, N. E.; Lenze, D.; Wansel, S.; Hummel, M.; Bornfeld, N.; Thiel, E.; Foerster, M. H.; et al. Significance of Gene Expression Analysis in Uveal Melanoma in Comparison to Standard Risk Factors for Risk Assessment of Subsequent Metastases. Eye Lond. Engl. 2008, 22, 997-1007, doi: 10.1038/sj.eye.6702779.
6 Worley, L. A.; Onken, M. D.; Person, E.; Robirds, D.; Branson, J.; Char, D. H.; Perry, A.; Harbour, J. W. Transcriptomic versus Chromosomal Prognostic Markers and Clinical Outcome in Uveal Melanoma. Clin. Cancer Res. 2007, 13, 1466-1471, doi: 10.1158/1078-0432.CCR-06-2401.
7. Cai, L.; Paez-Escamilla, M.; Walter, S. D.: Tarlan, B.; Decatur, C. L.; Perez, B. M.; Harbour, J. W. Gene Expression Profiling and PRAME Status Versus Tumor-Node-Metastasis Staging for Prognostication in Uveal Melanoma. Am. J. Ophthalmol. 2018, 195, 154-160, doi: 10.1016/j.ajo.2018.07.045.
8. Onken, M. D.; Worley, L. A.; Ehlers, J. P.; Harbour, J. W. Gene Expression Profiling in Uveal Melanoma Reveals Two Molec-ular Classes and Predicts Metastatic Death. Cancer Res. 2004, 64, 7205-7209, doi: 10.1158/0008-5472.CAN-04-1750.
9. Onken, M. D.; Worley, L. A.; Tuscan, M. D.; Harbour, J. W. An Accurate, Clinically Feasible Multi-Gene Expression Assay for Predicting Metastasis in Uveal Melanoma. J. Mol. Diagn. JMD 2010, 12, 461-468, doi: 10.2353/jmoldx.2010.090220.
10. Chappell, M. C.; Char, D. H.; Cole, T. B.; Harbour, J. W.; Mishra, K.; Weinberg, V. K.; Phillips, T. L. Uveal Melanoma: Molecular Pattern, Clinical Features, and Radiation Response. Am. J. Ophthalmol. 2012, 154, 227-232.e2, doi: 10.1016/j.ajo.2012.02.022.
11. Correa, Z. M.; Augsburger, J. J. Sufficiency of FNAB Aspirates of Posterior Uveal Melanoma for Cytologic versus GEP Clas-sification in 159 Patients, and Relative Prognostic Significance of These Classifications. Graefes Arch. Clin. Exp. Ophthalmol. Albrecht Von Graefes Arch. Klin. Exp. Ophthalmol. 2014, 252, 131-135, doi: 10.1007/s00417-013-2515-0.
12. Plasseraud, K. M.; Cook, R. W.; Tsai, T.; Shildkrot, Y.; Middlebrook, B.; Maetzold, D.; Wilkinson, J.; Stone, J.; Johnson, C.; Oelschlager, K.; et al. Clinical Performance and Management Outcomes with the DecisionDx-UM Gene Expression Profile Test in a Prospective Multicenter Study. J. Oncol. 2016, 2016, 5325762, doi: 10.1155/2016/5325762.
13. Walter, S. D.; Chao, D. L.; Feuer, W.; Schiffman, J.; Char, D. H.; Harbour, J. W. Prognostic Implications of Tumor Diameter in Association With Gene Expression Profile for Uveal Melanoma. JAMA Ophthalmol. 2016, 134, 734-740, doi: 10.1001/jamaophthalmol.2016.0913.
14. Plasseraud, K. M.; Wilkinson, J. K.; Oelschlager, K. M.; Poteet, T. M.; Cook, R. W.; Stone, J. F.; Monzon, F. A. Gene Expression Profiling in Uveal Melanoma: Technical Reliability and Correlation of Molecular Class with Pathologic Characteristics. Diagn. Pathol. 2017, 12, 59, doi: 10.1186/s13000-017-0650-3.
15. Aaberg, T. M.; Cook, R. W.; Oelschlager, K.; Maetzold, D.; Rao, P. K.; Mason, J. O. Current Clinical Practice: Differential Management of Uveal Melanoma in the Era of Molecular Tumor Analyses. Clin. Ophthalmol. Auckl. NZ 2014, 8, 2449-2460, doi: 10.2147/OPTH.S70839.
16. Davanzo, J. M.; Binkley, E. M.; Bena, J. F.; Singh, A. D. Risk-Stratified Systemic Surveillance in Uveal Melanoma. Br. J. Oph-thalmol. 2019, 103, 1868-1871, doi: 10.1136/bjophthalmol-2018-313569.
17. Velez, G.; Nguyen, H. V.; Chemudupati, T.; Ludwig, C. A.; Toral, M.; Reddy, S.; Mruthyunjaya, P.; Mahajan, V. B. Liquid Biopsy Proteomics of Uveal Melanoma Reveals Biomarkers Associated with Metastatic Risk. Mol. Cancer 2021, 20, 39, doi: 10.1186/s12943-021-01336-4.
18. Augsburger, J. J.; Corrêa, Z. M.; Augsburger, B. D. Frequency and Implications of Discordant Gene Expression Profile Class in Posterior Uveal Melanomas Sampled by Fine Needle Aspiration Biopsy. Am. J. Ophthalmol. 2015, 159, 248-256, doi: 10.1016/j.ajo.2014.10.026.
19. Heiferman, M. J.; Mahajan, V. B.; Mruthyunjaya, P. Proteomics in Uveal Melanoma. Curr. Opin. Ophthalmol. 2022, 33, 202-210, doi: 10.1097/ICU.0000000000000835.
20. Wierenga, A. P. A.; Cao, J.; Mouthaan, H.; van Weeghel, C.; Verdijk, R. M.; van Duinen, S. G.; Kroes, W. G. M.; Dogrusöz, M.; Marinkovic, M.; van der Burg, S. S. H.; et al. Aqueous Humor Biomarkers Identify Three Prognostic Groups in Uveal Mel-anoma. Invest. Ophthalmol. Vis. Sci. 2019, 60, 4740-4747, doi: 10.1167/iovs.19-28309.
21. Im, D.; Peng, C.-C.; Xu, L.; Kim, M. E.; Ostrow, D.; Yellapantula, V.; Bootwalla, M.; Biegel, J. A.; Gai, X.; Kuhn, P.; et al. Potential of Aqueous Humor as a Liquid Biopsy for Uveal Melanoma. Invest. Ophthalmol. Vis. Sci. 2022, 63, 1458.
1. Jager, M. J.; Shields, C. L.; Cebulla, C. M.; Abdel-Rahman, M. H.; Grossniklaus, H. E.; Stern, M.-H.; Carvajal, R. D.; Bel-fort, R. N.; Jia, R.; Shields, J. A.; et al. Uveal Melanoma. Nat. Rev. Dis. Primer 2020, 6, 24, doi: 10.1038/s41572-020-0158-0.
2. Kujala, E.; Mäkitie, T.; Kivelä, T. Very Long-Term Prognosis of Patients with Malignant Uveal Melanoma. Invest. Ophthalmol. Vis. Sci. 2003, 44, 4651-4659, doi: 10.1167/iovs.03-0538.
3. Onken, M. D.; Worley, L. A.; Char, D. H.; Augsburger, J. J.; Correa, Z. M.; Nudleman, E.; Aaberg, T. M.; Altaweel, M. M.; Bardenstein, D. S.; Finger, P. T.; et al. Collaborative Ocular Oncology Group Report No. 1: Prospective Validation of a Mul-ti-Gene Prognostic Assay in Uveal Melanoma. Ophthalmology 2012, 119, 1596-1603, doi: 10.1016/j.ophtha.2012.02.017.
4. Corrêa, Z. M.; Augsburger, J. J. Independent Prognostic Significance of Gene Expression Profile Class and Largest Basal Diameter of Posterior Uveal Melanomas. Am. J. Ophthalmol. 2016, 162, 20-27.e1, doi: 10.1016/j.ajo.2015.11.019.
5. Petrausch, U.; Martus, P.; Tönnies, H.; Bechrakis, N. E.; Lenze, D.; Wansel, S.; Hummel, M.; Bornfeld, N.; Thiel, E.; Foerster, M. H.; et al. Significance of Gene Expression Analysis in Uveal Melanoma in Comparison to Standard Risk Factors for Risk Assessment of Subsequent Metastases. Eye Lond. Engl. 2008, 22, 997-1007, doi: 10.1038/sj.eye.6702779.
6. Worley, L. A.; Onken, M. D.; Person, E.; Robirds, D.; Branson, J.; Char, D. H.; Perry, A.; Harbour, J. W. Transcriptomic versus Chromosomal Prognostic Markers and Clinical Outcome in Uveal Melanoma. Clin. Cancer Res. 2007, 13, 1466-1471, doi: 10.1158/1078-0432.CCR-06-2401.
7. Cai, L.; Paez-Escamilla, M.; Walter, S. D.; Tarlan, B.; Decatur, C. L.; Perez, B. M.; Harbour, J. W. Gene Expression Profiling and PRAME Status Versus Tumor-Node-Metastasis Staging for Prognostication in Uveal Melanoma. Am. J. Ophthalmol. 2018, 195, 154-160, doi: 10.1016/j.ajo.2018.07.045.
8. Onken, M. D.; Worley, L. A.; Ehlers, J. P.; Harbour, J. W. Gene Expression Profiling in Uveal Melanoma Reveals Two Molecular Classes and Predicts Metastatic Death. Cancer Res. 2004, 64, 7205-7209, doi: 10.1158/0008-5472.CAN-04-1750.
9. Onken, M. D.; Worley, L. A.; Tuscan, M. D.; Harbour, J. W. An Accurate, Clinically Feasible Multi-Gene Expression Assay for Predicting Metastasis in Uveal Melanoma. J. Mol. Diagn. JMD 2010, 12, 461-468, doi: 10.2353/jmoldx.2010.090220.
10. Chappell, M. C.; Char, D. H.; Cole, T. B.; Harbour, J. W.; Mishra, K.; Weinberg, V. K.; Phillips, T. L. Uveal Melanoma: Molecular Pattern, Clinical Features, and Radiation Response. Am. J. Ophthalmol. 2012, 154, 227-232.e2, doi: 10.1016/j.ajo.2012.02.022.
11. Correa, Z. M.; Augsburger, J. J. Sufficiency of FNAB Aspirates of Posterior Uveal Melanoma for Cytologic versus GEP Clas-sification in 159 Patients, and Relative Prognostic Significance of These Classifications. Graefes Arch. Clin. Exp. Ophthalmol. Albrecht Von Graefes Arch. Klin. Exp. Ophthalmol. 2014, 252, 131-135, doi: 10.1007/s00417-013-2515-0.
12. Plasseraud, K. M.; Cook, R. W.; Tsai, T.; Shildkrot, Y.; Middlebrook, B.; Maetzold, D.; Wilkinson, J.; Stone, J.; Johnson, C.; Oelschlager, K.; et al. Clinical Performance and Management Outcomes with the DecisionDx-UM Gene Expression Profile Test in a Prospective Multicenter Study. J. Oncol. 2016, 2016, 5325762, doi: 10.1155/2016/5325762.
13. Walter, S. D.; Chao, D. L.; Feuer, W.; Schiffman, J.; Char, D. H.; Harbour, J. W. Prognostic Implications of Tumor Diameter in Association With Gene Expression Profile for Uveal Melanoma. JAMA Ophthalmol. 2016, 134, 734-740, doi: 10.1001/jamaophthalmol.2016.0913.
14. Plasseraud, K. M.; Wilkinson, J. K.; Oelschlager, K. M.; Poteet, T. M.; Cook, R. W.; Stone, J. F.; Monzon, F. A. Gene Expression Profiling in Uveal Melanoma: Technical Reliability and Correlation of Molecular Class with Pathologic Characteristics. Diagn. Pathol. 2017, 12, 59, doi: 10.1186/s13000-017-0650-3.
15. Aaberg, T. M.; Cook, R. W.; Oelschlager, K.; Maetzold, D.; Rao, P. K.; Mason, J. O. Current Clinical Practice: Differential Management of Uveal Melanoma in the Era of Molecular Tumor Analyses. Clin. Ophthalmol. Auckl. NZ 2014, 8, 2449-2460, doi: 10.2147/OPTH.S70839.
16. Davanzo, J. M.; Binkley, E. M.; Bena, J. F.; Singh, A. D. Risk-Stratified Systemic Surveillance in Uveal Melanoma. Br. J. Oph-thalmol. 2019, 103, 1868-1871, doi: 10.1136/bjophthalmol-2018-313569.
17. Velez, G.; Nguyen, H. V.; Chemudupati, T.; Ludwig, C. A.; Toral, M.; Reddy, S.; Mruthyunjaya, P.; Mahajan, V. B. Liquid Biopsy Proteomics of Uveal Melanoma Reveals Biomarkers Associated with Metastatic Risk. Mol. Cancer 2021, 20, 39, doi: 10.1186/s12943-021-01336-4.
18. Augsburger, J. J.; Corrêa, Z. M.; Augsburger, B. D. Frequency and Implications of Discordant Gene Expression Profile Class in Posterior Uveal Melanomas Sampled by Fine Needle Aspiration Biopsy. Am. J. Ophthalmol. 2015, 159, 248-256, doi: 10.1016/j.ajo.2014.10.026.
19. Heiferman, M. J.; Mahajan, V. B.; Mruthyunjaya, P. Proteomics in Uveal Melanoma. Curr. Opin. Ophthalmol. 2022, 33, 202-210, doi: 10.1097/ICU.0000000000000835.
20. Wierenga, A. P. A.; Cao, J.; Mouthaan, H.; van Weeghel, C.; Verdijk, R. M.; van Duinen, S. G.; Kroes, W. G. M.; Dogrusöz, M.; Marinkovic, M.; van der Burg, S. S. H.; et al. Aqueous Humor Biomarkers Identify Three Prognostic Groups in Uveal Mel-anoma. Invest. Ophthalmol. Vis. Sci. 2019, 60, 4740-4747, doi: 10.1167/iovs. 19-28309.
21. Im, D.; Peng, C.-C.; Xu, L.; Kim, M. E.; Ostrow, D.; Yellapantula, V.; Bootwalla, M.; Biegel, J. A.; Gai, X.; Kuhn, P.; et al. Potential of Aqueous Humor as a Liquid Biopsy for Uveal Melanoma. Invest. Ophthalmol. Vis. Sci. 2022, 63, 1458.
22. Lamas, N. J.; Martel, A.; Nahon-Estève, S.; Goffinet, S.; Macocco, A.; Bertolotto, C.; Lassalle, S.; Hofman, P. Prognostic Bi-omarkers in Uveal Melanoma: The Status Quo, Recent Advances and Future Directions. Cancers 2021, 14, 96, doi: 10.3390/cancers14010096.
23. Robertson, A. G.; Shih, J.; Yau, C.; Gibb, E. A.; Oba, J.; Mungall, K. L.; Hess, J. M.; Uzunangelov, V.; Walter, V.; Danilova, L.; et al. Integrative Analysis Identifies Four Molecular and Clinical Subsets in Uveal Melanoma. Cancer Cell 2017, 32, 204-220.e15, doi: 10.1016/j.ccell.2017.07.003.
24. Bakhoum, M. F.; Esmaeli, B. Molecular Characteristics of Uveal Melanoma: Insights from the Cancer Genome Atlas (TCGA) Project. Cancers 2019, 11, 1061, doi: 10.3390/cancers11081061.
25. Gelfo, V.; Romaniello, D.; Mazzeschi, M.; Sgarzi, M.; Grilli, G.; Morselli, A.; Manzan, B.; Rihawi, K.; Lauriola, M. Roles of IL-1 in Cancer: From Tumor Progression to Resistance to Targeted Therapies. Int. J. Mol. Sci. 2020, 21, doi: 10.3390/ijms21176009.
26. Stuckel, A. J.; Zeng, S.; Lyu, Z.; Zhang, W.; Zhang, X.; Dougherty, U.; Mustafi, R.; Zhang, Q.; Joshi, T.; Bissonnette, M.; et al. Epigenetic DNA Modifications Upregulate SPRY2 in Human Colorectal Cancers. Cells 2021, 10, 2632, doi: 10.3390/cells10102632.
27. Berry, J. L.; Xu, L.; Kooi, I.; Murphree, A. L.; Prabakar, R. K.; Reid, M.; Stachelek, K.; Le, B. H. A.; Welter, L.; Reiser, B. J.; et al. Genomic CIDNA Analysis of Aqueous Humor in Retinoblastoma Predicts Eye Salvage: The Surrogate Tumor Biopsy for Retinoblastoma. Mol. Cancer Res. MCR 2018, 16, 1701-1712, doi: 10.1158/1541-7786.MCR-18-0369.

All publications, patents, and patent applications, Genbank sequences, websites and other published materials referred to throughout the disclosure herein are herein incorporated by reference to the same extent as if each individual publication, patent, or patent application, Genbank sequences, websites and other published materials was specifically and individually indicated to be incorporated by reference. In the event that the definition of a term incorporated by reference conflicts with a term defined herein, this specification shall control.

1. A method to stratify risk of metastasis in a uveal melanoma (UM) patient to class 1 (low metastatic risk) or class 2 (high metastatic risk) comprising providing an aqueous humor (AH) liquid biopsy sample from said patient and determining modulation of expression of one or more genes in said sample, wherein said modulation of expression of said one or more genes is correlated with class 1 or class 2.

2. A method to determine an increased risk or likelihood of metastasis in a uveal melanoma (UM) patient comprising providing an aqueous humor (AH) liquid biopsy sample from said patient and determining modulation of expression of one or more genes in said sample, wherein said modulation of expression of said one or more genes is correlated with class 2, high metastatic risk.

3. A method to stratify risk of metastasis in a uveal melanoma (UM) patient to class 1 (low metastatic risk) or class 2 (high metastatic risk) comprising providing an aqueous humor (AH) liquid biopsy sample from said patient and determining modulation of expression of one or more genes in said sample, wherein said modulation of expression of said one or more genes is correlated with class 1 (low metastatic risk) or class 2 (high metastatic risk).

4. The method of claim 1, wherein said one or more genes are NUCB2, DBI, CSTB, CD14, SCG2, GNLY, RGMB, CLPS, CXCL10, TGFBR3, IGFBP3, CTSF, VWA1, SERPINE1, HGF, IL18BP, MSR1, TREM2, TNFRSF1B, PLAUR, OSCAR, PILRA, TNFRSF11A, ANGPTL4, SMAD5, TNFRSF12A, SMOC2, TNFRSF1A, ST6GAL1, LAIR1, CD74, DPP7, MAPT, TDGF1, PMVK, CNTN2, AMN, SLC39A5, DGKZ, AXIN1, CEBPB, ZBTB16, CHI3L1, DUOX2 or CST3.

5. The method of claim 1, wherein one or more of NUCB2, DBI, CSTB, CD14, SCG2, GNLY, RGMB, CLPS, CXCL10, TGFBR3, IGFBP3, CTSF, VWA1, SERPINE1, HGF, IL18BP, MSR1, TREM2, TNFRSF1B, PLAUR, OSCAR, PILRA, TNFRSF11A, ANGPTL4, SMAD5, TNFRSF12A, SMOC2, TNFRSF1A, ST6GAL1, LAIR1 or CD74 genes are upregulated for class 2.

6. The method of claim 1, wherein one or more of DPP7, MAPT, TDGF1, PMVK, CNTN2, AMN, SLC39A5, DGKZ, AXIN1, CEBPB, ZBTB16, CHI3L1, DUOX2 or CST3 genes are downregulated for class 2.

7. The method of claim 1, wherein the one or more genes are human genes.

8. The method of claim 1, wherein UM tumors present in said subject are too small to provide biopsied tissue.

9. The method of claim 1, further comprising treating said subject for UM and/or UM metastases.

10. The method of claim 9, wherein the treatment is surgery, radiation, photocoagulation, chemotherapy, immunotherapy and/or thermotherapy.

11. The method of claim 2, wherein said one or more genes are NUCB2, DBI, CSTB, CD14, SCG2, GNLY, RGMB, CLPS, CXCL10, TGFBR3, IGFBP3, CTSF, VWA1, SERPINE1, HGF, IL18BP, MSR1, TREM2, TNFRSF1B, PLAUR, OSCAR, PILRA, TNFRSF11A, ANGPTL4, SMAD5, TNFRSF12A, SMOC2, TNFRSF1A, ST6GAL1, LAIR1, CD74, DPP7, MAPT, TDGF1, PMVK, CNTN2, AMN, SLC39A5, DGKZ, AXIN1, CEBPB, ZBTB16, CHI3L1, DUOX2 or CST3.

12. The method of claim 2, wherein one or more of NUCB2, DBI, CSTB, CD14, SCG2, GNLY, RGMB, CLPS, CXCL10, TGFBR3, IGFBP3, CTSF, VWA1, SERPINE1, HGF, IL18BP, MSR1, TREM2, TNFRSF1B, PLAUR, OSCAR, PILRA, TNFRSF11A, ANGPTL4, SMAD5, TNFRSF12A, SMOC2, TNFRSF1A, ST6GAL1, LAIR1 or CD74 genes are upregulated for class 2.

13. The method of claim 2, wherein one or more of DPP7, MAPT, TDGF1, PMVK, CNTN2, AMN, SLC39A5, DGKZ, AXIN1, CEBPB, ZBTB16, CHI3L1, DUOX2 or CST3 genes are downregulated for class 2.

14. The method of claim 3, wherein said one or more genes are NUCB2, DBI, CSTB, CD14, SCG2, GNLY, RGMB, CLPS, CXCL10, TGFBR3, IGFBP3, CTSF, VWA1, SERPINE1, HGF, IL18BP, MSR1, TREM2, TNFRSF1B, PLAUR, OSCAR, PILRA, TNFRSF11A, ANGPTL4, SMAD5, TNFRSF12A, SMOC2, TNFRSF1A, ST6GAL1, LAIR1, CD74, DPP7, MAPT, TDGF1, PMVK, CNTN2, AMN, SLC39A5, DGKZ, AXIN1, CEBPB, ZBTB16, CHI3L1, DUOX2 or CST3.

15. The method of claim 3, wherein one or more of NUCB2, DBI, CSTB, CD14, SCG2, GNLY, RGMB, CLPS, CXCL10, TGFBR3, IGFBP3, CTSF, VWA1, SERPINE1, HGF, IL18BP, MSR1, TREM2, TNFRSF1B, PLAUR, OSCAR, PILRA, TNFRSF11A, ANGPTLA, SMAD5, TNFRSF12A, SMOC2, TNFRSF1A, ST6GAL1, LAIR1 or CD74 genes are upregulated for class 2.

16. The method of claim 3, wherein one or more of DPP7, MAPT, TDGF1, PMVK, CNTN2, AMN, SLC39A5, DGKZ, AXIN1, CEBPB, ZBTB16, CHI3L1, DUOX2 or CST3 genes are downregulated for class 2.

17. The method of claim 2, further comprising treating said subject for UM and/or UM metastases.

18. The method of claim 17, wherein the treatment is surgery, radiation, photocoagulation, chemotherapy, immunotherapy and/or thermotherapy.

19. The method of claim 3, further comprising treating said subject for UM and/or UM metastases.

20. The method of claim 19, wherein the treatment is surgery, radiation, photocoagulation, chemotherapy, immunotherapy and/or thermotherapy.
